(19) United States
(12) Patent Application Publication
(10) Pub. No.: US 2010/0280067 A1
(43) Pub. Date: Nov. 4, 2010

(54) INHIBITORS OF ACETYL-COA CARBOXYLASE

(76) Inventors: Pakala Kumara Savithru SARMA, New Bowenpally (IN); Vinod Parameshwaran Acharya, Makarpura (IN); Srinivas Rao Kasibhatla, San Diego, CA (US); Vellarkad Narayana Viswanadhan, Jakkur Plantation (IN); Polisetti Shekhar, Nereducharla (IN); Alexander Bischoff, Smithtown, NY (US)

Correspondence Address: Forest Laboratories, Inc.;Attn: Charles S. Ryan, 500 COMMACK ROAD, Commack, NY, 11725, US

(21) Appl. No.: 12/771,019

(22) Filed: Apr. 30, 2010

Related U.S. Application Data
(60) Provisional application No. 61/174,152, filed on Apr. 30, 2009.
(60) Provisional application No. 61/247,620, filed on Oct. 1, 2009.

(51) Int. Cl. A61K 31/472; C07D 217/14; C07D 217/24; A61P 3/10; A61P 3/04; A61P 9/00

(52) U.S. Cl. 514/309; 546/146; 514/307; 546/141

(57) ABSTRACT

The present invention relates to compounds that act as acetyl-CoA carboxylase (ACC) inhibitors. The invention also relates to methods of preparing the compounds, compositions containing the compounds, and to methods of treatment using the compounds.

This application claims the benefit of U.S. Provisional Application Ser. No. 61/174,152, filed Apr. 30, 2009 and U.S. Provisional Application Ser. No. 61/247,620, filed Oct. 1, 2009, the entire disclosures of each of which are hereby incorporated by reference.

### FIELD OF THE INVENTION

The present invention relates to compounds which are useful, for example, for the prevention or treatment of type 2 diabetes, obesity, atherosclerosis and cardiovascular diseases in humans mediated through acetyl-CoA carboxylase (ACC). Processes for the preparation of described compounds, pharmaceutical compositions containing the described compounds, and methods for treating diseases mediated through acetyl-CoA carboxylase are also provided.

### BACKGROUND OF THE INVENTION

Metabolic syndromes are associated with several diseases and disorders, such as obesity, diabetes, and diabesity (typically defined by the occurrence in a single patient of both diabetes and obesity or other overweight conditions, and characterized by elevated blood glucose levels). Metabolic syndromes are typically defined by a clustering of cardiovascular risk factors that increase the risks of coronary heart disease and/or type II diabetes. Such metabolic syndromes are often characterized by elevated insulin concentration, and are often associated with such conditions as visceral obesity, hyperlipidemia, atherogenic dyslipidemia, hyperglycemia, hypertension, hyperurecemia and renal dysfunction. Metabolic syndromes, together with insulin resistance, are increasingly viewed as being major causes of type II diabetes and atherosclerosis.

Recent studies have suggested that abnormal fatty acid metabolism is a contributing cause of metabolic syndrome (see Wakil et al., Fatty acid metabolism: Target for metabolic syndrome, J. Lipid Res. 50, S138-S143, April 2009; as well as Kusunoki et al., Modulation of fatty acid metabolism as a potential approach to the treatment of obesity and the metabolic syndrome, Endocrine 1, 91-100, Feb. 29 2006).

Abnormal fatty acid synthesis has also been found to be a cause for obesity, as well as nonalcoholic fatty liver disease (NAFLD) and liver dysfunction (such as NAFLD-associated liver dysfunction). Prevalence of NAFLD has markedly increased in the recent years (Cusi K., Nonalcoholic fatty liver disease in type 2 diabetes mellitus, Curr. Opin. Endocrinol. Diabetes Obes. 16(2), 141-9, April 2009).

Acetyl-CoA carboxylase, a member of biotin-dependent carboxylases family, catalyzes the formation of malonyl-CoA, an intermediate that regulates fatty acid biosynthesis and oxidation. ACC exists as two different isoenzymes, ACC1 and ACC2. Both forms exhibit high sequence homology except at the N-terminal ends.

There are several differences between ACC1 and ACC2. For example, ACC2, a 2458 amino acid peptide, contains a 114 amino acid portion that facilitates anchoring of ACC to the mitochondrial membrane. In contrast, ACC1 lacks this targeting sequence and thereby remains cytosolic. In addition, the ACC1 and ACC2 isoforms also exhibit divergent tissue expression profiles, providing the basis for different functions. In particular, in oxidative tissues (such as heart and skeletal muscles), ACC2 forms malonyl-CoA which mainly regulates fatty acid oxidation through inhibition of carnityl palmitoyltransferase 1 (CPT-1) inhibition. In the lipogeneic tissues, such as liver and adipose tissues, malonyl-CoA produced by ACC1 is utilized as a substrate for fatty acid synthesis and chain elongation.

Accordingly, there remains a need for the development of acetyl-CoA carboxylase (ACC) inhibitors that can be used for the treatment of type 2 diabetes, obesity, diabesity, NAFLD, liver dysfunction disorders, atherosclerosis, cardiovascular diseases mediated through ACC, and combinations of these diseases and disorders.

### SUMMARY OF THE INVENTION

The present invention relates to compounds that act as inhibitors of acetyl-CoA carboxylase. The invention also relates to methods of preparing the compounds, compositions containing the compounds, and to methods of treatment using the compounds.

### DETAILED DESCRIPTION OF THE INVENTION

In one embodiment, compounds of Formula 1 are provided:

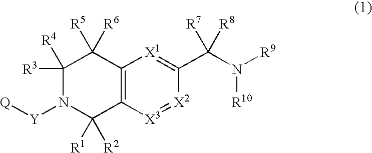

wherein:

each X¹, X², and X³ are each, independently, CR¹¹ or nitrogen;

Y is a direct bond, —C(O)—, —O—, —(CR¹²R¹³)ₘ—, —NR²⁰—, or —S(O)ₙ—;

Q is selected from the group consisting of aryl, heteroaryl, cycloalkyl, heterocycloalkyl or heterocycloalkenyl group; wherein aryl, heteroaryl, cycloalkyl, heterocycloalkyl and heterocycloalkenyl is optionally substituted with one or more substituents selected from the group consisting of halogen, alkyl, heteroalkyl, —CN, —NO₂, —Si(R¹¹)₃, —S(O)ₙR¹¹, —C(O)H, —C(O)R¹⁴, —NR¹¹R¹⁴, C(O)OR¹⁴, NHS(O)ₙCH₃, OR¹¹, SR¹⁴, lower alkoxy, fluoro substituted lower alkoxy, lower alkylthio, fluoro substituted lower alkylthio, and wherein said aryl, heteroaryl, cycloalkyl, and heterocycloalkyl groups are optionally fused with or covalently bound to other aryl, heteroaryl, cycloalkyl, or heterocycloalkyl groups to form a polycyclic ring system having 2 to 4 rings;

R¹, R², R³, R⁴, R⁵, R⁶, R¹², and R¹³ are, independently, selected from the group consisting of hydrogen, halogen, alkyl, heteroalkyl, cycloalkyl, heterocycloalkyl, arylalkyl, heteroarylalkyl, —CN, —OR¹⁵, —NO₂, —NR¹¹R¹⁴, SiR²⁰₃, —S(O)ₙR¹⁶, —C(O)H, and —C(O)R¹⁷;

R⁷ and R⁸ are each, independently, selected from the group consisting of hydrogen, halogen, alkyl, heteroalkyl, cycloalkyl, heterocycloalkyl, —CN, —OR¹⁵, —NO₂, —SiR²⁰₃, —NR⁹R¹⁰, —S(O)ₙR¹⁶, —C(O)H, and —C(O)R¹⁷;

R⁹ and R¹⁰ are each, independently, selected from the group consisting of hydrogen, alkyl, heteroalkyl, cycloalkyl, heterocycloalkyl, aryl, heteroaryl, arylalkyl, heteroarylalkyl, —S(O)ₙR¹⁶, —C(O)H, —C(O)R¹⁷, and —NR¹⁸R¹⁹;

R¹¹ is hydrogen, halogen, alkylene, alkyl, heteroalkyl, cycloalkyl, heterocycloalkyl, —CN, —OR¹⁵, —NO₂, —SiR²⁰₃, —N(R¹⁴)₂, —S(O)ₙR¹⁶, —C(O)H, and —C(O)R¹⁷, wherein alkylene is optionally substituted with one or more substituents selected from alkyl, cycloalkyl, heteroalkyl, heteroaryl, heterocycloalkyl, —N(R¹⁴)ₙ, —OR¹⁴, or —C(O)OR¹⁴;

R¹⁴ is alkyl, alkoxy, heteroalkyl, cycloalkyl, heterocycloalkyl, aryl, heteroaryl, arylalkyl, heteroarylalkyl, —C(O)H, —C(O)R¹⁷, or —NR¹⁸R¹⁹;

R¹⁵ can be the same or different and is selected from the group consisting of hydrogen, alkyl, heteroalkyl, cycloalkyl, heterocycloalkyl, aryl, heteroaryl, arylalkyl, heteroarylalkyl, and —P(O)₂OR²⁰;

R¹⁶ is alkyl, heteroalkyl, cycloalkyl, heterocycloalkyl, aryl, heteroaryl, arylalkyl, or heteroarylalkyl;

R¹⁷ is —OR¹⁶, —SR¹⁶, —NR¹⁸R¹⁹, alkyl, heteroalkyl, cycloalkyl, heterocycloalkyl, aryl, heteroaryl, arylalkyl, or heteroarylalkyl;

each R¹⁸ and R¹⁹ is, independently, selected from the group consisting of hydrogen, alkyl, heteroalkyl, cycloalkyl, heterocycloalkyl, aryl, heteroaryl, arylalkyl, and heteroarylalkyl;

R²⁰ is hydrogen, alkyl, heteroalkyl, cycloalkyl, heterocycloalkyl, aryl, heteroaryl, arylalkyl, or heteroarylalkyl;

m is 1 or 2; and

each n is, independently 0, 1, or 2;

or optical isomers, pharmaceutically acceptable salts, solvates or N-oxides thereof;

with the proviso that at least one of R⁷ and R⁸ is not both hydrogen when Y is —(CR¹²R¹³)ₘ—.

In another embodiment, compounds of Formula 1 are provided:

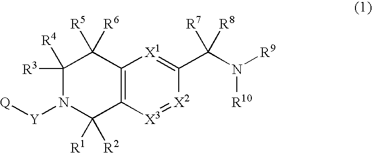

wherein:

each X¹, X², and X³ are each, independently, CR¹¹ or nitrogen;

Y is a direct bond, —C(O)—, —O—, —(CR¹²R¹³)ₘ—, —NR²⁰—, or —S(O)ₙ—;

Q is selected from the group consisting of aryl, heteroaryl, cycloalkyl, heterocycloalkyl or heterocycloalkenyl group; wherein aryl, heteroaryl, cycloalkyl, heterocycloalkyl and heterocycloalkenyl is optionally substituted with one or more substituents selected from the group consisting of halogen, alkyl, heteroalkyl, —CN, —NO₂, —Si(R¹¹)₃, —S(O)ₙR¹¹, —C(O)H, —C(O)R¹⁴, —NR¹¹R¹⁴, C(O)OR¹⁴, NHS(O)ₙCH₃, OR¹¹, SR¹⁴, lower alkoxy, fluoro substituted lower alkoxy, lower alkylthio, fluoro substituted lower alkylthio, and wherein said aryl, heteroaryl, cycloalkyl, and heterocycloalkyl groups are optionally fused with or covalently bound to other aryl, heteroaryl, cycloalkyl, or heterocycloalkyl groups to form a polycyclic ring system having 2 to 4 rings;

R¹, R², R³, R⁴, R⁵, R⁶, R¹², and R¹³ are, independently, selected from the group consisting of hydrogen, halogen, alkyl, heteroalkyl, cycloalkyl, heterocycloalkyl, arylalkyl, heteroarylalkyl, —CN, —OR¹⁵, —NO₂, —NR¹¹R¹⁴, —SiR²⁰₃, —S(O)ₙR¹⁶, —C(O)H, and —C(O)R¹⁷; and wherein geminal R¹, R², R³, R⁴, R⁵, R⁶, R¹², or R¹³ groups can, independently, form carbonyl, thiocarbonyl, spiro-cyclopropyl, substituted imines or oximes with the carbon atom to which they are bound;

R⁷ and R⁸ are each, independently, selected from the group consisting of hydrogen, halogen, alkyl, heteroalkyl, cycloalkyl, heterocycloalkyl, —CN, —OR¹⁵, —NO₂, —SiR²⁰₃, NR¹¹R¹⁴, —S(O)ₙR¹⁶, —C(O)H, and —C(O)R¹⁷; wherein geminal R⁷ and R⁸ groups can, independently, form spiro-cyclopropyl with the carbon atom to which they are bound; and wherein R⁷ and R⁸ do not collectively form a carbonyl with the carbon atom to which they are bound;

R⁹ and R¹⁰ are each, independently, selected from the group consisting of hydrogen, alkyl, heteroalkyl, cycloalkyl, heterocycloalkyl, aryl, heteroaryl, arylalkyl, heteroarylalkyl, —S(O)ₙR¹⁶, —C(O)H, —C(O)R¹⁷, and —NR¹⁸R¹⁹; wherein R⁹ and R¹⁰ groups can form heteroaryl groups with the nitrogen atom to which they are bound; wherein R⁹ and R¹⁰ groups can form heterocycloalkyl groups with the nitrogen atom to which they are bound when R⁷ and R⁸ are not both hydrogen; wherein R⁹ and R¹⁰ groups can, independently, form heterocycloalkyl groups with the nitrogen atom to which they are bound when at least one of X¹, X², or X³ is nitrogen; wherein R⁹ is not —CH(alkyl)C(O)NH₂ when R¹⁰ is —S(O)ₙR¹⁶ and n is 2 and R¹⁶ is a substituted or unsubstituted phenyl or thiophene group; and wherein R⁹ is not methyl when R¹⁰ is pyrrolidin-3-yl;

R¹¹ is hydrogen, halogen, alkylene, alkyl, heteroalkyl, cycloalkyl, heterocycloalkyl, —CN, —OR¹⁵, —NO₂, —SiR²⁰₃, —N(R¹⁴)ₙ, —S(O)ₙR¹⁶, —C(O)H, or —C(O)R¹⁷, wherein alkylene is optionally substituted with one or more substituents selected from the group consisting of alkyl, cycloalkyl, heteroalkyl, heteroaryl, heterocycloalkyl, —N(R¹⁴)ₙ, —OR¹⁴, and —C(O)OR¹⁴;

R¹⁴ is alkyl, alkoxy, heteroalkyl, cycloalkyl, heterocycloalkyl, aryl, heteroaryl, arylalkyl, heteroarylalkyl, —C(O)H, —C(O)R¹⁷, or —NR¹⁸R¹⁹;

R¹⁵ can be the same or different and is hydrogen, alkyl, heteroalkyl, cycloalkyl, heterocycloalkyl, aryl, heteroaryl, arylalkyl, heteroarylalkyl, or —P(O)₂OR²⁰;

R¹⁶ is alkyl, heteroalkyl, cycloalkyl, heterocycloalkyl, aryl, heteroaryl, arylalkyl, or heteroarylalkyl;

R¹⁷ is —OR¹⁶, —SR¹⁶, —NR¹⁸R¹⁹, alkyl, heteroalkyl, cycloalkyl, heterocycloalkyl, aryl, heteroaryl, arylalkyl, or heteroarylalkyl;

each R¹⁸ and R¹⁹ are, independently, selected from the group consisting of hydrogen, alkyl, heteroalkyl, cycloalkyl, heterocycloalkyl, aryl, heteroaryl, arylalkyl, and heteroarylalkyl; and wherein geminal R¹⁸ and R¹⁹ groups can, independently, form heteroaryl or heterocycloalkyl groups with the nitrogen atom to which they are bound;

R²⁰ is hydrogen, alkyl, heteroalkyl, cycloalkyl, heterocycloalkyl, aryl, heteroaryl, arylalkyl, or heteroarylalkyl;

m is 1 or 2; and

each n is, independently 0, 1, or 2;

or optical isomers, pharmaceutically acceptable salts, solvates or N-oxides thereof;

with the proviso that at least one of R⁷ and R⁸ is not both hydrogen when Y is —(CR¹²R¹³)ₘ—.

In one embodiment, compounds of Formula 1 are provided wherein Q is an aryl group, wherein said aryl is optionally substituted with one or more substituents selected from the group consisting halogen, alkyl, heteroalkyl, —CN, —NO₂, —Si(R¹¹)₃, —S(O)ₙR¹¹, —C(O)H, —C(O)R¹⁴, —NR¹¹R¹⁴, C(O)OR¹⁴, NHS(O)ₙCH₃, OR¹¹, SR¹⁴, lower alkoxy, fluoro substituted lower alkoxy, lower alkylthio, and fluoro substituted lower alkylthio.

In another embodiment, compounds of formula 1 are provided, wherein Q is selected from the group consisting of:

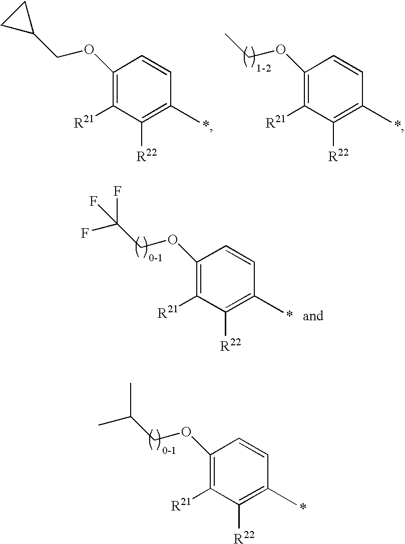

wherein, R²¹, R²² are independently selected from the group consisting of alkyl, —NO₂, fluoro substituted lower alkoxy and halogen.

In yet another embodiment, compounds of formula 1 are provided, wherein Q is a heteroaryl group, wherein said heteroaryl is optionally substituted with one or more substituents selected from the group consisting of halogen, alkyl, heteroalkyl, —CN, —NO₂, —Si(R¹¹)₃, —S(O)ₙR¹¹, —C(O)H, —C(O)R¹⁴, —NR¹¹R¹⁴, C(O)OR¹⁴, NHS(O)ₙCH₃, OR¹¹, SR¹⁴, lower alkoxy, fluoro substituted lower alkoxy, lower alkylthio, and fluoro substituted lower alkylthio.

In yet another embodiment, compounds of formula 1 are provided, wherein Q is a cycloalkyl group, wherein said cycloalkyl is optionally substituted with one or more substituents selected from the group consisting of halogen, alkyl, heteroalkyl, —CN, —NO₂, —Si(R¹¹)₃, —S(O)ₙR¹¹, —C(O)H, —C(O)R¹⁴, —NR¹¹R¹⁴, C(O)OR¹⁴, NHS(O)ₙCH₃, OR¹¹, SR¹⁴, lower alkoxy, fluoro substituted lower alkoxy, lower alkylthio, and fluoro substituted lower alkylthio.

In another embodiment, compounds of formula 1 are provided, wherein said Q is a heterocycloalkyl group, wherein said heterocycloalkyl is optionally substituted with one or more substituents selected from the group consisting of halogen, alkyl, heteroalkyl, —CN, —NO₂, —Si(R¹¹)₃, —S(O)ₙR¹¹, —C(O)H, —C(O)R¹⁴, —NR C(O)OR¹⁴, NHS(O)ₙCH₃, OR¹¹, SR¹⁴, lower alkoxy, fluoro substituted lower alkoxy, lower alkylthio, and fluoro substituted lower alkylthio.

In one embodiment, compounds of Formula 1 are provided wherein X¹, X², and X³ are CR¹¹.

In one embodiment, compounds of Formula 1 are provided wherein Y is —C(O)— or —CH₂—.

In yet another embodiment, compounds of Formula 1 are provided, wherein Y is —CH₂—, Q is an aryl group, wherein said aryl is optionally substituted with one or more substituents selected from the group consisting of halogen, alkyl, heteroalkyl, —CN, —NO₂, —Si(R¹¹)₃, S(O)ₙR¹¹, C(O)H, —C(O)R¹⁴, —NR¹¹R¹⁴, C(O)OR¹⁴, NHS(O)ₙCH₃, OR¹¹, SR¹⁴, lower alkoxy, fluoro substituted lower alkoxy, lower alkylthio, and fluoro substituted lower alkylthio.

In another embodiment, compounds of Formula 1 are provided, wherein Y is —CH₂—, and Q is heteroaryl, cycloalkyl, heterocycloalkyl or heterocycloalkenyl group, wherein said aryl, heteroaryl, cycloalkyl, heterocycloalkyl, and heterocycloalkenyl is not unsubstituted.

In one embodiment, a compound of Formula 1 is provided wherein Y is —O—, —NR²⁰—, or —S(O)ₙ—.

In one embodiment, a compound of Formula 1 is provided wherein, when m is 2, then (CR¹²R¹³)ₘ is optionally a 3-, 4-, or 5-membered carbocycle selected from:

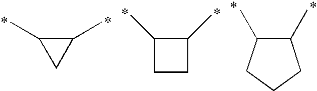

and said carbocycle is optionally substituted by one or more groups that may be the same or different and which are, independently, selected from the group consisting of alkyl, halogen, —CN, —OR¹⁵, —SiR²⁰₃, and —NO₂.

In some embodiments, compounds of Formula 1 are provided, when Y is —C(O)— or —CH₂C(O)—, Q is not a substituted or unsubstituted pyrrolidin-1-yl, piperidin-1-yl, azepan-1-yl, azocan-1-yl, piperazin-1-yl, 1,4-diazepan-1-yl, or 1,4-diazocan-1-yl.

In some embodiments, compounds of Formula 1 are provided, when Y is a direct bond, then R⁷ and R⁸ are not both hydrogen.

In some embodiments, compounds of Formula 1 are provided, wherein when m is 2, then R⁷ and R⁸ are not both hydrogen.

In some embodiments, compounds of Formula 1 are provided, wherein geminal R¹, R², R³, R⁴, R⁵, R⁶, R¹², or R¹³ groups, independently, form carbonyl, thiocarbonyl, spiro-cyclopropyl, substituted imines (imines in the form of —C(═NR¹⁴)—), or oximes (oximes in the form of —C(═N—OR²⁰)—) with the carbon atom to which they are bound.

In some embodiments, compounds of Formula 1 are provided, wherein geminal R⁷ and R⁸ groups, independently, form spiro-cyclopropyl with the carbon atom to which they are bound.

In some embodiments, compounds of Formula 1 are provided, wherein R⁷ and R⁸ do not collectively form a carbonyl with the carbon atom to which they are bound.

In some embodiments, compounds of Formula 1 are provided, wherein R⁹ and R¹⁰ groups form heteroaryl groups with the nitrogen atom to which they are bound.

In some embodiments, compounds of Formula 1 are provided, wherein R⁹ and R¹⁰ groups form heterocycloalkyl groups with the nitrogen atom to which they are bound when R⁷ and R⁸ are not both hydrogen.

In some embodiments, compounds of Formula 1 are provided, wherein R⁹ and R¹⁰ groups, independently, form heterocycloalkyl groups with the nitrogen atom to which they are bound when at least one of X¹, X², or X³ is nitrogen;

In some embodiments, compounds of Formula 1 are provided, wherein R⁹ is not —CH(alkyl)C(O)NH₂ when R¹⁰ is —S(O)ₙR¹⁶, n is 2, and R¹⁶ is a substituted or unsubstituted phenyl or thiophene group.

In some embodiments, compounds of Formula 1 are provided, wherein R⁹ is not methyl when R¹⁰ is pyrrolidin-3-yl.

In some embodiments, compounds of Formula 1 are provided, wherein geminal R¹⁸ and R¹⁹ groups, independently, form heteroaryl or heterocycloalkyl groups with the nitrogen atom to which they are bound.

In one embodiment, the compound of Formula 1 is selected from the group consisting of:
N-{1-[2-(4-Cyclopropylmethoxybenzoyl)-1,2,3,4-tetrahydroisoquinolin-6-yl]ethyl}acetamide,
N-{1-[2-(4-Cyclopropylmethoxybenzyl)-1,2,3,4-tetrahydroisoquinolin-6-yl]ethyl}acetamide,
N-{1-[2-(4-Cyclopropylmethoxybenzyl)-1,2,3,4-tetrahydroisoquinolin-6-yl]ethyl}propionamide,
{1-[2-(4-Cyclopropylmethoxybenzyl)-1,2,3,4-tetrahydroisoquinolin-6-yl]ethyl}carbamic acid methyl ester,
{1-[2-(4-Cyclopropylmethoxybenzyl)-1,2,3,4-tetrahydroisoquinolin-6-yl]ethyl}carbamic acid ethyl ester,
N-{1-[2-(3-Chloro-4-propoxybenzyl)-1,2,3,4-tetrahydroisoquinolin-6-yl]ethyl}acetamide,
N-{1-[2-(3-Chloro-4-isobutoxybenzyl)-1,2,3,4-tetrahydroisoquinolin-6-yl]ethyl}acetamide,
N-{1-[2-(3-Chloro-4-isobutoxybenzyl)-1,2,3,4-tetrahydroisoquinolin-6-yl]ethyl}acetamide,
N-{1-[2-(3-Chloro-4-cyclopropylmethoxybenzyl)-1,2,3,4-tetrahydroisoquinolin-6-yl]ethyl}acetamide,
N-{1-[2-(3-Chloro-4-ethoxybenzyl)-1,2,3,4-tetrahydroisoquinolin-6-yl]ethyl}acetamide,
N-{1-[2-(3-Bromo-4-isopropoxybenzyl)-1,2,3,4-tetrahydroisoquinolin-6-yl]ethyl}acetamide,
N-{1-[2-(3-Bromo-4-propoxybenzyl)-1,2,3,4-tetrahydroisoquinolin-6-yl]ethyl}acetamide,
N-{1-[2-(3-Bromo-4-ethoxybenzyl)-1,2,3,4-tetrahydroisoquinolin-6-yl]ethyl}acetamide,
N-{1-[2-(3-Bromo-4-cyclopropylmethoxybenzyl)-1,2,3,4-tetrahydroisoquinolin-6-yl]ethyl}acetamide,
N-{1-[2-(3-Bromo-4-isobutoxybenzyl)-1,2,3,4-tetrahydroisoquinolin-6-yl]ethyl}acetamide,
N-{1-[2-(4-Isopropoxy-2-methylbenzyl)-1,2,3,4-tetrahydroisoquinolin-6-yl]ethyl}acetamide,
N-{1-[2-(4-Ethoxy-2-methylbenzyl)-1,2,3,4-tetrahydroisoquinolin-6-yl]ethyl}acetamide,
N-{1-[2-(2-Methyl-4-propoxybenzyl)-1,2,3,4-tetrahydroisoquinolin-6-yl]ethyl}acetamide,
4-[2-(4-Cyclopropylmethoxy-2-methylbenzyl)-1,2,3,4-tetrahydroisoquinolin-6-yl]-pentan-2-one,
N-{1-[2-(4-isopropoxy-2-methylbenzyl)-1,2,3,4-tetrahydroisoquinolin-6-yl]ethyl}acetamide,
N-{1-[2-(2-Bromo-4-isopropoxybenzyl)-1,2,3,4-tetrahydroisoquinolin-6-yl]ethyl}acetamide,
N-{1-[2-(2-Chloro-4-isopropoxybenzyl)-1,2,3,4-tetrahydroisoquinolin-6-yl]ethyl}acetamide,
N-{1-[2-(4-Cyclopropylmethoxybenzyl)-1-oxo-1,2,3,4-tetrahydroisoquinolin-6-yl]ethyl}acetamide,
N-(1-{2-[2-Chloro-4-(2,2,2-trifluoro-ethoxy)-benzyl]-1,2,3,4-tetrahydro-isoquinolin-6-yl}-ethyl)-acetamide,
N-(1-{2-[2-Nitro-4-(2,2,2-trifluoro-ethoxy)-benzyl]-1,2,3,4-tetrahydro-isoquinolin-6-yl}-ethyl)-acetamide,
N-{1-[2-(4-Ethoxy-2-nitro-benzyl)-1,2,3,4-tetrahydro-isoquinolin-6-yl]-ethyl}-acetamide, and
N-{1-[2-(4-cyclopropylmethoxy-3-trifluoromethoxybenzyl)-1,2,3,4-tetrahydroisoquinolin-6-yl]ethyl}acetamide,

or optical isomers, pharmaceutically acceptable salts, solvates or N-oxides thereof;

wherein free base forms listed above can also be in the form of a pharmaceutically acceptable salt,

wherein a compound listed above (in either a free base form or in the form of a pharmaceutically acceptable salt) can also be in the form of a solvate (such as a hydrate),

wherein a compound listed above (in either a free base form or in the form of a pharmaceutically acceptable salt) can also be in the form of an N-oxide,

wherein a compound listed above (in a free base form or solvate or N-oxide thereof, or in the form of a pharmaceutically acceptable salt or solvate thereof) can also be in the form of a polymorph, and

wherein if the compound exhibits chirality it can be in the form of a mixture of enantiomers such as a racemate or a mixture of diastereomers, or can be in the form of a single enantiomer or a single diastereomer.

As used herein the term “halogen” means, unless otherwise stated, a fluorine, chlorine, bromine, or iodine atom.

As used herein, the suffix “ene” added to any of the described terms means that the substituent is connected to two other parts in the compound. For example, “alkylene” is (CH₂)ₙ. Said alkylene can be optionally substituted by one or more groups that may be the same or different and which can be conceptually formed from an alkylene by replacing the hydrogen atom in alkylene with another atom or substituent group. In some embodiments of the invention, the substituents are alkyl, cycloalkyl, heteroalkyl, heteroaryl, heterocycloalkyl, —NH₂, —NHR′, N(R′)₂, OR′, or —C(O)OR′, wherein each occurrence of R′ is independently selected from alkyl, heteroalkyl, cycloalkyl, heteroalkyl, aryl, heteroaryl, arylalkyl, and heteroarylalkyl; and wherein each R′ is optionally substituted by one or two more groups, independently selected from halogen, —R′, —OR′, —OH, —SH, —SR′, —NO₂, —CN, —C(O)R′, —OC(O)R′, —CON(R′)₂, or —OC(O)N(R′)₂, —NH₂, —NHR′, —N(R′)₂, —NHCOR′, —NHCOH, —NHCONH₂, —NHCONHR′, —NHCON(R′)₂, —NRCOR′, —NRCOH, —NHCO₂H, —NHCO₂R′, —CO₂R′, —CO₂H, —CHO, —CONH₂, —CONHR′, —CON(R′)₂, —S(O)₂H, —S(O)₂R′, —SO₂NH₂, —S(O)H, —S(O)R′, —SO₂NHR′, —SO₂N(R)₂, —NHS(O)₂H, —NR′S(O)₂H, —NHS(O)₂R′, —NR′S(O)₂R′, or —Si(R′)₃.

The term “alkyl”, by itself or as part of another substituent, means, unless otherwise stated, a straight chain or branched chain, or cyclic hydrocarbon radical, or combination thereof, which may be fully saturated, monounsaturated or polyunsaturated and can include divalent and multivalent radicals, having the number of not more than 15 of carbon atoms. Examples of saturated hydrocarbon radicals include, but are not limited to groups such as methyl, ethyl, n-propyl, isopropyl, n-butyl, t-butyl, isobutyl, sec-butyl, cyclopropyl, pentyl, hexyl, heptyl, octyl, nonyl, decyl, undecyl, and dodecyl, 2-(cyclopropyl)ethyl, cyclohexylmethyl, cyclopropylethyl, cyclohexyl, cyclopropylmethyl, 1-methylbutyl, 2-methylbutyl, 3-methylbutyl, 1,1-dimethylpropyl, 1,2-dimethylpropyl, 2,2-dimethylpropyl, 1-ethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, ethylmethylpropyl, trimethylpropyl, methylhexyl, dimethylpentyl, ethylpentyl, ethylmethylbutyl, dimethylbutyl, spiropentyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to vinyl, prop-2-enyl, crotyl, isopent-2-enyl, butadien-2-yl, penta-2,4-dienyl, penta-1,4-dien-3-yl, ethynyl, prop-1-ynyl, prop-3-ynyl, but-3-ynyl, and the higher homologs and isomers, and the like.

The term “heteroalkyl”, by itself or as part of another substituent, means, unless otherwise stated, a straight or branched chain, or cyclic hydrocarbon radical, or combinations thereof, consisting of one to fourteen carbon atoms and from one to six heteroatoms selected from oxygen, nitrogen, sulfur, and silicon, and wherein the nitrogen, sulfur and silicon atoms may optionally be oxidized and the nitrogen atom may optionally be quaternized. The heteroatoms O, N and S may be placed at any interior position of the heteroalkyl group. The heteroatom Si may be placed at any position of the heteroalkyl group, including the position at which the heteroalkyl group is attached to the remainder of the molecule. Examples include, but are not limited to 2-methoxyethyl, 2-(methylamino)ethyl, 2-(dimethylamino)ethyl, 2-(ethylthio)methyl, 2-(methylsulfinyl)ethyl, 2-(methylsulfonyl)ethyl, 2-methoxyvinyl, trimethylsilyl, dimethyl(vinyl)silyl, 2-(cyclopropylthio)ethyl, and 2-(methoxyimino)ethyl. Up to two heteroatoms may be consecutive, such as, for example, (methoxyamino)methyl and trimethylsilyloxy.

The term “heteroalkenyl”, by itself or as part of another substituent, means, unless otherwise stated, a straight or branched chain, or cyclic hydrocarbon radical, or combinations thereof, unsaturated with at least one double bond, consisting of one to fourteen carbon atoms and from one to six heteroatoms selected from oxygen, nitrogen, sulfur, and silicon, and wherein the nitrogen, sulfur and silicon atoms may optionally be oxidized and the nitrogen atom may optionally be quaternized. The heteroatoms O, N and S may be placed at any interior position of the heteroalkenyl group. The heteroatom Si may be placed at any position of the heteroalkenyl group, including the position at which the heteroalkenyl group is attached to the remainder of the molecule.

The terms “cycloalkyl”, “heterocycloalkyl” and “heterocycloalkenyl”, by themselves or as part of another substituent, represent, unless otherwise stated, cyclic versions of “alkyl”, “heteroalkyl”, and “heteroalkenyl” respectively. Additionally, for heterocycloalkyl, and heterocycloalkenyl, a heteroatom can occupy the position at which the heterocycle is attached to the remainder of the molecule. Examples of cycloalkyl include, but are not limited to cyclopropyl, cyclopentyl, cyclohexyl, cyclohex-1-enyl, cyclohex-3-enyl, cycloheptyl, cyclooctyl, norbornyl, decalinyl, adamant-1-yl, adamant-2-yl, bicyclo[2.1.0]pentyl, bicyclo[3.1.0]hexyl, spiro[2.4]heptyl, spiro[2.5]octyl, bicyclo[5.1.0]octyl, spiro[2.6]nonyl, bicyclo[2.2.0]hexyl, spiro[3.3]heptyl, bicyclo[4.2.0]octyl, and spiro[3.5]nonyl, and the like. Examples of heterocycloalkyl include, but are not limited to piperidinyl, piperidin-2-yl, piperidin-3-yl, morpholin-4-yl, morpholin-3-yl, tetrahydrofuran-2-yl, tetrahydrofuran-3-yl, tetrahydrothien-2-yl, tetrahydrothien-3-yl, piperazinyl, piperazin-2-yl, and the like.

The term “alkoxy” refers to those alkyl groups attached to the remainder of the molecule via an oxygen atom. Suitable examples of alkoxy groups include, but are not limited to methoxy, ethoxy, n-propoxy, iso-propoxy, n-butoxy, tert-butoxy, pentoxy, hexoxy, heptoxy, and the like.

The term “lower alkoxy” refers to “C1 to C7 alkoxy” such as methoxy, ethoxy, n-propoxy, iso-propoxy, n-butoxy, tert-butoxy, and the like. “C1 to C7 alkoxy” can be optionally substituted, meaning that the alkyl portion of the alkoxy can be substituted to form, for example, branched alkoxy, and the like.

The term “lower alkylthio” refers to “C1 to C7 alkylthio” such as methylthio, ethylthio, n-propylthio, isopropylthio, n-butylthio, t-butylthio and the like. ‘C1 to C7 alkylthio” can be optionally substituted, meaning that the alkyl portion of the alkylthio can be substituted to form, for example, fluoro substituted lower alkylthio, and the like.

The term “aryl” means, unless otherwise stated, a polyunsaturated, typically aromatic, hydrocarbon substituent which can be a monocyclic system or polycyclic ring system (with up to three rings) which are fused together or linked covalently. The monocyclic or polycyclic ring system comprises about 5 to about 16 carbon atoms. Suitable examples of aryl groups include, but are not limited to phenyl, naphthyl, anthracenyl, and the like.

The term “heteroaryl” refers to “aryl” groups that contain from one to four heteroatoms selected from nitrogen, oxygen, and sulfur, wherein the nitrogen and sulfur atoms are optionally oxidized, and one or several nitrogen atom are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl.

The terms “arylalkyl” and “heteroarylalkyl” is meant to include those radicals in which an aryl group is attached to an alkyl group (e.g., benzyl, phenethyl, pyridylmethyl, and the like) including those alkyl groups in which a carbon atom (e.g., a methylene group) has been replaced by, for example, an oxygen atom (e.g., phenoxymethyl, pyrid-2-yloxymethyl, 3-(naphth-1-yloxy)propyl, and the like).

Each of the above terms “alkyl”, “heteroalkyl”, “cycloalkyl”, “heterocycloalkyl”, “alkoxy”, “aryl”, “heteroaryl”, “arylalkyl”, and “heteroarylalkyl” are meant to include both substituted and unsubstituted forms of the indicated radical. Said “alkyl”, “heteroalkyl”, “cycloalkyl”, “heterocycloalkyl”, “alkoxy”, “aryl”, “heteroaryl”, “arylalkyl”, and “heteroarylalkyl” groups are optionally substituted by one or more groups that may be the same or different and which are, independently, selected from halogen (e.g., in the form of —CF₃, —CF₂CF₃, —CHF₂, —CH₂F, and the like), —R′, —OR′, —OH, —SH, —SR′, —NO₂, —CN, —C(O)R′, —OC(O)R′, —CON(R′)₂, or —OC(O)N(R′)₂, —NH₂, —NHR′, —N(R′)₂, —NHCOR′, —NHCOH, —NHCONH₂, —NHCONHR′, —NHCON(R′)₂, —NRCOR′, —NRCOH, —NHCO₂H, —NHCO₂R′, —CO₂R′, —CO₂H, —CHO, —CONH₂, —CONHR′, —CON(R′)₂, —S(O)₂H, —S(O)₂R′, —SO₂NH₂, —S(O)H, —S(O)R′, —SO₂NHR′, —SO₂N(R)₂, —NHS(O)₂H, —NR′S(O)₂H, —NHS(O)₂R′, —NR′S(O)₂R′, or —Si(R′)₃; and wherein a saturated carbon atom of said “alkyl”, “heteroalkyl”, “cycloalkyl”, “heterocycloalkyl”, “alkoxy”, “aryl”, “heteroaryl”, “arylalkyl”, and “heteroarylalkyl” groups is optionally substituted with one or more groups that may be the same or different and which are, independently, selected from ═O, ═S, ═NNHR′, ═NNH₂, ═NN(R′)₂, ═N—OR′, ═N—OH, ═NNHCOR′, ═NNHCOH, ═NNHCO₂R′, ═NNHCO₂H, ═NNHSO₂R′, ═NNHSO₂H, ═N—CN, ═NH, or ═NR; and wherein each occurrence of R′ is, independently, selected from alkyl, heteroalkyl, cycloalkyl, heterocycloalkyl, aryl, heteroaryl, arylalkyl, and heteroarylalkyl; and wherein each R′ is optionally substituted by one or two groups that may be the same or different and which are, independently, selected from halogen, —R′, —OR′, —OH, —SH, —SR′, —NO₂, —CN, —C(O)R′, —OC(O)R′, —CON(R′)₂, or —OC(O)N(R′)₂, —NH₂, —NHR′, —N(R′)₂, —NHCOR′, —NHCOH, —NHCONH₂, —NHCONHR′, —NHCON(R′)₂, —NRCOR′, —NRCOH, —NHCO₂H, —NHCO₂R′, —CO₂R′, —CO₂H, —CHO, —CONH₂, —CONHR′, —CON(R′)₂, —S(O)₂H, —S(O)₂R′, —SO₂NH₂, —S(O)H, —S(O)R′, —SO₂NHR′, —SO₂N(R)₂, —NHS(O)₂H, —NR′S(O)₂H, —NHS(O)₂R′, —NR′S(O)₂R′, or —Si(R′)₃; and wherein one ore two saturated carbon atoms of R′ are optionally substituted with one or more groups that may be the same or different and which are, independently, selected from ═O, ═S, ═NNHR′, ═NNH₂, ═NN(R′)₂, ═N—OR′, ═N—OH, ═NNHCOR′, ═NNHCOH, ═NNHCO₂R′, ═NNHCO₂H, ═NNHSO₂R′, ═NNHSO₂H, ═N—CN, ═NH, or ═NR′.

As used herein, the term “heteroatom” is meant to include oxygen (O), nitrogen (N), and sulfur (S).

The phrases “independently selected”, “independently”, and their variants, when used in reference to two or more of the same substituent group (e.g., two or more R¹⁵ groups within the same compound), are used herein to mean that that two or more groups can be the same or different. For example, the compound of Formula 1 can comprise two R¹⁵ groups, wherein one R¹⁵ group is hydrogen and the other R¹⁵ group is an alkyl. Moreover, the compound of Formula 1 can comprise two R¹⁵ groups, wherein both R¹⁵ groups are hydrogen.

When term “direct bond”, when used in reference to a particular component of the compound of Formula 1, can be absent from the compound. For example, when Y is defined as being a direct bond, then Q may be directly bound to the N to which Y is depicted as being bound.

One of ordinary skill in the art will recognize that compounds of Formula 1 can exist in different tautomeric and geometrical isomeric forms. All of these compounds, including cis-isomers, trans-isomers, E-isomers, Z-isomers, diastereomic mixtures, racemates, nonracemic mixtures of enantiomers, substantially pure, and pure enantiomers and diastereomers, are within the scope of the present invention. Substantially pure enantiomers contain no more than 5% w/w of the corresponding opposite enantiomer, preferably no more than 2%, most preferably no more than 1%.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, dibenzoyltartaric, ditoluoyltartaric and camphorsulfonic acid. Mixtures of diastereoisomers can be separated into their individual diastereomers on the basis of their physical and/or chemical differences by methods known to those skilled in the art, for example, by chromatography or fractional crystallization. The optically active bases or acids are then liberated from the separated diastereomeric salts. A different process for separation of optical isomers involves the use of chiral chromatography (e.g., chiral HPLC columns), with or without conventional derivation, optimally chosen to maximize the separation of the enantiomers. Suitable chiral HPLC columns are manufactured by Diacel, e.g., Chiracel OD and Chiracel OJ, among many others, all routinely selectable. Enzymatic separations, with or without derivitization, are also useful. The optically active compounds of Formula 1 can likewise be obtained by utilizing optically active starting materials in chiral synthesis processes under reaction conditions which do not cause racemization.

In addition, one of ordinary skill in the art will recognize that the compounds can be used in different enriched isotopic forms, e.g., enriched in the content of ²H, ³H, ¹¹C, ¹³C and/or ¹⁴C. In one particular embodiment, the compounds are deuterated. Such deuterated forms can be made the procedure described in U.S. Pat. Nos. 5,846,514 and 6,334,997. As described in U.S. Pat. Nos. 5,846,514 and 6,334,997, deuteration can improve the efficacy and increase the duration of action of drugs.

Deuterium substituted compounds can be synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10) (2000), 110 pp.; Kabalka, George W.; Varma, Rajender S., The synthesis of radiolabeled compounds via organometallic intermediates, Tetrahedron (1989), 45(21), 6601-21; Evans, E. Anthony, Synthesis of radiolabeled compounds, J. Radioanal. Chem. (1981), 64(1-2), 9-32.]

Where applicable, the present invention also relates to useful forms of the compounds as disclosed herein, such as base free forms, and pharmaceutically acceptable salts or prodrugs of all the compounds of the present invention for which salts or prodrugs can be prepared. Pharmaceutically acceptable salts include those obtained by reacting the main compound, functioning as a base with an inorganic or organic acid to form a salt, for example, salts of hydrochloric acid, sulfuric acid, phosphoric acid, methane sulfonic acid, camphor sulfonic acid, oxalic acid, maleic acid, succinic acid, citric acid, formic acid, hydrobromic acid, benzoic acid, tartaric acid, fumaric acid, salicylic acid, mandelic acid, and carbonic acid. Pharmaceutically acceptable salts also include those in which the main compound functions as an acid and is reacted with an appropriate base to form, e.g., sodium, potassium, calcium, magnesium, ammonium, and choline salts. Those skilled in the art will further recognize that acid addition salts of the claimed compounds may be prepared by reaction of the compounds with the appropriate inorganic or organic acid via any of a number of known methods. Alternatively, alkali and alkaline earth metal salts can be prepared by reacting the compounds of the invention with the appropriate base via a variety of known methods.

The following are further examples of acid salts that can be obtained by reaction with inorganic or organic acids: acetates, DIPEAtes, alginates, citrates, aspartates, benzoates, benzenesulfonates, bisulfates, butyrates, camphorates, digluconates, cyclopentanepropionates, dodecylsulfates, ethanesulfonates, glucoheptanoates, glycerophosphates, hemisulfates, heptanoates, hexanoates, fumarates, hydrobromides, hydroiodides, 2-hydroxyethanesulfonates, lactates, maleates, methanesulfonates, nicotinates, 2-naphthalenesulfonates, oxalates, palmoates, pectinates, persulfates, 3-phenylpropionates, picrates, pivalates, propionates, succinates, tartrates, thiocyanates, tosylates, mesylates and undecanoates.

For example, the pharmaceutically acceptable salt can be a hydrochloride, a hydrobromide, a hydroformate, or a maleate.

Preferably, the salts formed are pharmaceutically acceptable for administration to mammals. However, pharmaceutically unacceptable salts of the compounds are suitable as intermediates, for example, for isolating the compound as a salt and then converting the salt back to the free base compound by treatment with an alkaline reagent. The free base can then, if desired, be converted to a pharmaceutically acceptable acid addition salt.

One of ordinary skill in the art will also recognize that some of the compounds of Formula 1 can exist in different polymorphic forms. As known in the art, polymorphism is an ability of a compound to crystallize as more than one distinct crystalline or “polymorphic” species. A polymorph is a solid crystalline phase of a compound with at least two different arrangements or polymorphic forms of that compound molecule in the solid state. Polymorphic forms of any given compound are defined by the same chemical formula or composition and are as distinct in chemical structure as crystalline structures of two different chemical compounds.

One of ordinary skill in the art will further recognize that compounds of Formula 1 can exist in different solvate forms. Solvates of the compounds of the invention may also form when solvent molecules are incorporated into the crystalline lattice structure of the compound molecule during the crystallization process.

The present invention also includes prodrugs of compounds of Formula 1. The term prodrug is intended to represent covalently bonded carriers, which are capable of releasing the active ingredient of Formula 1 when the prodrug is administered to a mammalian subject. Release of the active ingredient occurs in vivo. Prodrugs can be prepared by techniques known to one skilled in the art. These techniques generally modify appropriate functional groups in a given compound. These modified functional groups however regenerate original functional groups by routine manipulation or in vivo. Prodrugs of compounds of Formula 1 include compounds wherein a hydroxy, amino, carboxylic, or a similar group is modified. Examples of prodrugs include, but are not limited to esters (e.g., acetate, formate, and benzoate derivatives), carbamates (e.g., N,N-dimethylaminocarbonyl) of hydroxy or amino functional groups in compounds of Formula 1), amides (e.g., trifluoroacetylamino, acetylamino, and the like), and the like. Prodrugs of compounds of Formula 1 are also within the scope of this invention.

The present invention also provides processes for preparing the compounds of Formula 1. Suitable general reaction schemes are shown below.

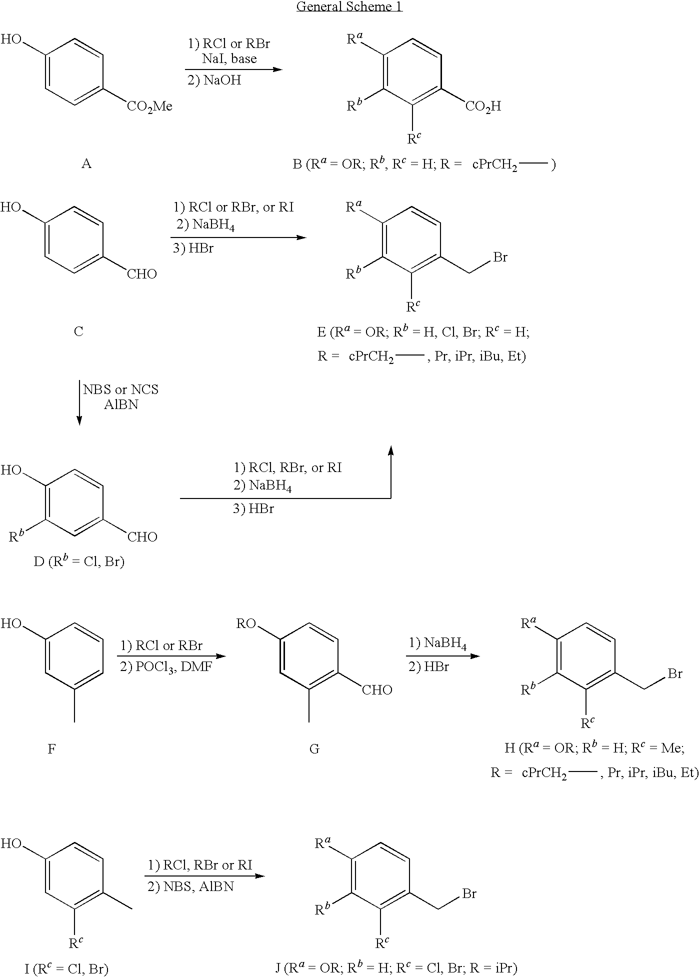

Commercially available ester A may be O-alkylated with an appropriate alkyl halide in the presence of a suitable base and can subsequently be subjected to a standard hydrolysis procedure known to the one skilled in the art to generate carboxylic acid B.

Commercially available benzaldehyde C may be meta-halogenated using a suitable halogenating agent (such as NBS in the presence of AIBN) known to the one skilled in the art. O-Alkylation can be achieved with an appropriate alkyl halide in the presence of a suitable base the reduction of the aldehyde can be carried out under standard aldehyde reduction conditions known to the one skilled in the art (e.g., with NaBH₄). Conversion of the resulting alcohol to the corresponding halide can be achieved with a hydrohalogenic acid under standard S_{N}1 substitution conditions known to the one skilled in the art to give benzyl halide E.

Commercially available cresol F can be O-alkylated with an appropriate alkyl halide in the presence of a suitable base. The aromatic ring may be ortho-formylated under Vilsmeier-Haack reaction conditions known to the one skilled in the art to render benzaldehyde G. Reduction of the aldehyde under standard aldehyde reduction conditions known to the one skilled in the art may be followed by conversion of the resulting alcohol to the corresponding halide with a hydrohalogenic acid under standard S_{N}1 substitution conditions known to the one skilled in the art to give benzyl halide H.

Commercially available cresol F can be O-alkylated with an appropriate alkyl halide in the presence of a suitable base and the methyl group can be halogenated by means of an agent (such as NBS in the presence of AIBN) known to the one skilled in the art to generate benzyl halide J.

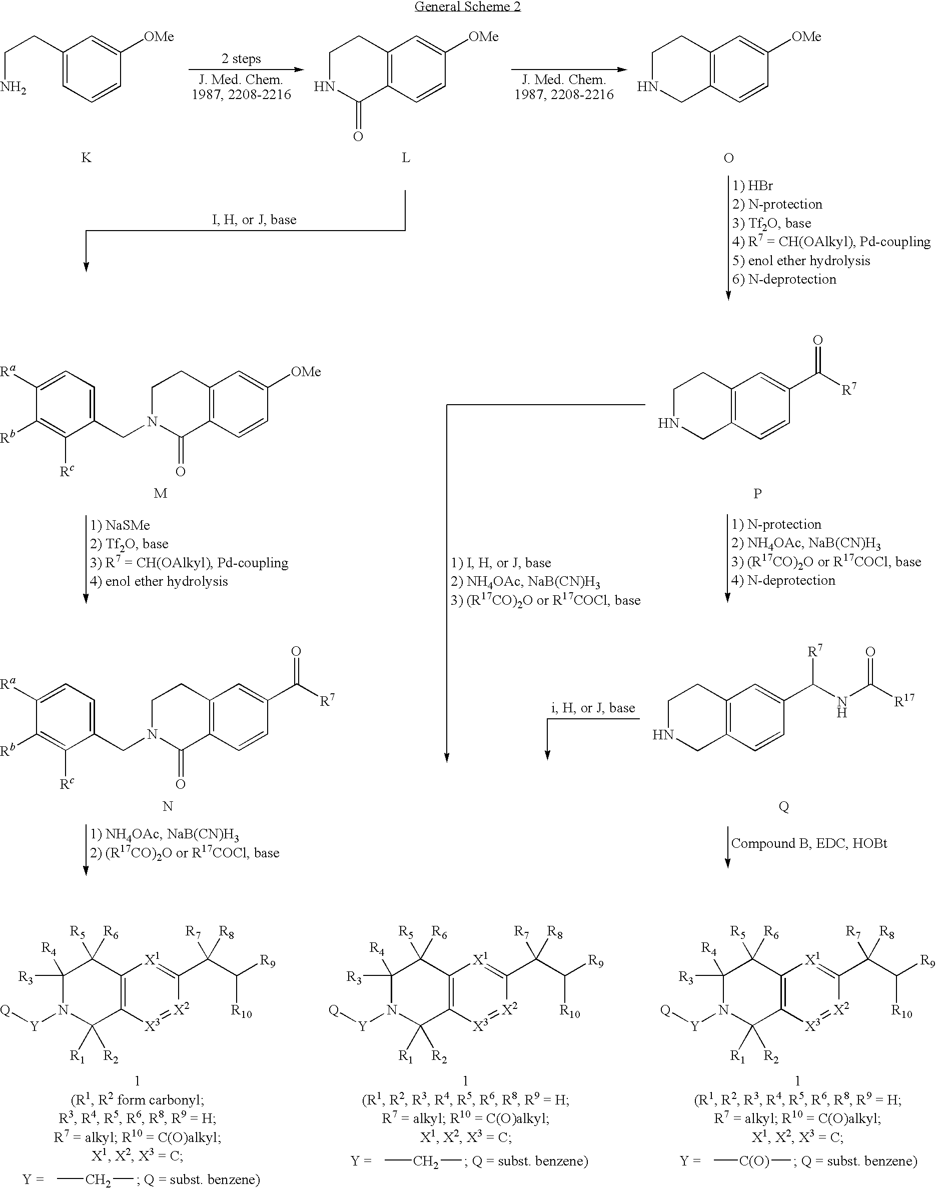

Commercially available 2-(3-methoxyphenyl)ethanamine K can be converted to 3,4-dihydroisoquinolin-1(2H)-one L in two steps according to the procedure reported in J. Med. Chem. 1987, vol. 30, p. 2208-2216. The amine of 3,4-dihydroisoquinolin-1(2H)-one L may be reacted with benzyl halide I, H, or J in the presence of a suitable base under standard substitution conditions known to one skilled in the art to afford N-substituted 3,4-dihydroisoquinolin-1(2H)-one M. O-Demethylation may be carried out with a suitable agent (such as sodium methanethiolate) known to one skilled in the art. Activation of the resulting hydroxy group by means of conversion to a triflate group may be followed by coupling with a vinyl ether derivative (such as alkenyloxybutane) under Heck coupling reaction conditions in the presence of a suitable catalyst and base known to one skilled in the art. An alkylcabonyl substituted 3,4-dihydroisoquinolin-1(2H)-one may be obtained by subsequent hydrolysis of the resulting enol ether with a suitable acid (such as hydrochloric acid) known to one skilled in the art to generate compound N. Conversion of the so obtained ketone may be carried out under reductive amination reaction conditions using a suitable combination of an imine forming agent (such as ammonium acetate) and a suitable reducing agent (such as NaBH₃CN) known to one skilled in the art. The resulting amine may be reacted with an appropriate acylating agent (such as an carboxylic acid anhydride or acyl chloride) known to one skilled in the art in the presence of a suitable base and under standard acylating reaction conditions to furnish a compound of Formula 1.

Compound L can be converted compound O according to the procedure reported in J. Med. Chem. 1987, vol. 30, p. 2208-2216. O-Demethylation may be carried out with a suitable agent (such as hydrobromic acid) known to one skilled in the art. The secondary amine may be protected with a Boc, benzyl or Cbz group via standard conditions known to one of ordinary skill. Activation of the resulting hydroxy group by means of conversion to a triflate group may be followed by coupling with a vinyl ether derivative (such as alkenyloxybutane) under Heck coupling reaction conditions in the presence of a suitable catalyst and base known to one skilled in the art. An alkylcabonyl substituted 3,4-dihydroisoquinolin-1(2H)-one may be obtained by subsequent hydrolysis of the resulting enol ether with a suitable acid (such as hydrochloric acid) known to one skilled in the art. If the N-protecting group is acid labile, then it may have be removed simultaneously in the previous step. If the N-protecting group is not acid labile it can be removed via standard conditions known to one of ordinary skill to render compound P. The amine of compound P may be reacted with benzyl halide I, H, or J in the presence of a suitable base under standard substitution conditions known to one skilled in the art.

Conversion of the ketone may be carried out under reductive amination reaction conditions using a suitable combination of an imine forming agent (such as ammonium acetate) and a suitable reducing agent (such as NaBH₃CN) known to one skilled in the art. The resulting amine may be reacted with an appropriate acylating agent (such as an carboxylic acid anhydride or acyl chloride) known to one skilled in the art in the presence of a suitable base and under standard acylating reaction conditions to furnish a compound of Formula 1.

The secondary amine of compound P may be protected with a Boc, benzyl or Cbz group via standard conditions known to one of ordinary skill. Conversion of the ketone may be carried out under reductive amination reaction conditions using a suitable combination of an imine forming agent (such as ammonium acetate) and a suitable reducing agent (such as NaBH₃CN) known to one skilled in the art. The resulting amine may be reacted with an appropriate acylating agent (such as an carboxylic acid anhydride or acyl chloride) known to one skilled in the art in the presence of a suitable base and under standard acylating reaction conditions known to one skilled in the art. The N-protecting group can be removed via standard conditions known to one of ordinary skill to render compound Q. Compound Q may be converted to a compound of Formula 1 by reaction with benzyl halide I, H, or J in the presence of a suitable base under standard substitution conditions known to one skilled in the art or, alternatively, by coupling the amine with an appropriately substituted carboxylic acid (B) in the presence of a standard peptide coupling reagent (such as EDC) or an appropriately substituted carboxylic acid chloride to give the desired amide product.

The compounds of the invention can be administered alone or as an active ingredient of a formulation. Thus, the present invention also includes pharmaceutical compositions of compounds of formula 1, containing, for example, one or more pharmaceutically acceptable carriers.

Numerous standard references are available that describe procedures for preparing various formulations suitable for administering the compounds according to the invention. Examples of potential formulations and preparations are contained, for example, in the Handbook of Pharmaceutical Excipients, American Pharmaceutical Association (current edition); Pharmaceutical Dosage Forms: Tablets (Lieberman, Lachman and Schwartz, editors) current edition, published by Marcel Dekker, Inc., as well as Remington's Pharmaceutical Sciences (Arthur Osol, editor), 1553-1593 (current edition).

Administration of the compounds of the present invention may be accomplished according to patient needs, for example, orally, nasally, parenterally (subcutaneously, intraveneously, intramuscularly, intrasternally and by infusion) by inhalation, rectally, vaginally, topically and by ocular administration.

Various solid oral dosage forms can be used for administering compounds of the invention including such solid forms as tablets, gelcaps, capsules, caplets, granules, lozenges and bulk powders. The compounds of the present invention can be administered alone or combined with various pharmaceutically acceptable carriers, diluents (such as sucrose, mannitol, lactose, starches) and excipients known in the art, including but not limited to suspending agents, solubilizers, buffering agents, binders, disintegrants, preservatives, colorants, flavorants, lubricants and the like. Time release capsules, tablets and gels are also advantageous in administering the compounds of the present invention.

Various liquid oral dosage forms can also be used for administering compounds of the inventions, including aqueous and non-aqueous solutions, emulsions, suspensions, syrups, and elixirs. Such dosage forms can also contain suitable inert diluents known in the art such as water and suitable excipients known in the art such as preservatives, wetting agents, sweeteners, flavorants, as well as agents for emulsifying and/or suspending the compounds of the invention. The compounds of the present invention may be injected, for example, intravenously, in the form of an isotonic sterile solution. Other preparations are also possible.

Suppositories for rectal administration of the compounds of the present invention can be prepared by mixing the compound with a suitable excipient such as cocoa butter, salicylates and polyethylene glycols. Formulations for vaginal administration can be in the form of a pessary, tampon, cream, gel, past foam, or spray formula containing, in addition to the active ingredient, such suitable carriers as are known in the art.

For topical administration the pharmaceutical composition can be in the form of creams, ointments, liniments, lotions, emulsions, suspensions, gels, solutions, pastes, powders, sprays, and drops suitable for administration to the skin, eye, ear or nose. Topical administration may also involve transdermal administration via means such as transdermal patches.

Aerosol formulations suitable for administering via inhalation also can be made. For example, the compounds of Formula 1 can be administered by inhalation in the form of a powder (e.g., micronized) or in the form of atomized solutions or suspensions. The aerosol formulation can be placed into a pressurized acceptable propellant.

### Methods of Treatment

The compounds of the present invention may be useful as inhibitors of acetyl-CoA carboxylase (ACC) enzymes, for example ACC1 and ACC2, or alternatively, ACC1 or ACC2. Therefore, the compounds are useful in the treatment of conditions mediated by ACC1 and ACC2, or alternatively, ACC1 or ACC2 enzymes.

According to another embodiment, the present invention relates to a method of treating a disease or condition mediated by acetyl-CoA carboxylase enzymes by administering to a patient in need thereof a therapeutically effective amount of a compound of Formula 1.

An ACC-mediated disease or condition includes but is not limited to a disease or condition which is, or is related to, cardiovascular disease, dyslipidemias (including but not limited to disorders of serum levels of triglycerides, hypertriglyceridemia, VLDL, HDL, LDL, cholesterol, total cholesterol, hypercholesterolemia, as well as cholesterol disorders (including disorders characterized by defective reverse cholesterol transport), familial combined hyperlipidemia, coronary artery disease, atherosclerosis, heart disease, cerebrovascular disease (including, but not limited to stroke, ischemic stroke and transient ischemic attack (TIA), peripheral vascular disease, and ischemic retinopathy. In an embodiment, compounds of the invention will, in a patient, increase HDL levels and/or decrease triglyceride levels and/or decrease LDL or non-HDL-cholesterol levels.

An ACC-mediated disease or condition also includes metabolic syndrome (including but not limited to dyslipidemia, obesity and insulin resistance, hypertension, microalbuminemia, hyperuricaemia, and hypercoagulability), Syndrome X, diabetes, pre-diabetes, insulin resistance, decreased glucose tolerance, non-insulin-dependent diabetes mellitus, Type II diabetes, Type I diabetes, diabetic complications (such as diabetic retinopathy, neuropathy, and nephropathy), body weight disorders (including but not limited to obesity, overweight, cachexia and anorexia), weight loss, body mass index and leptin related diseases. In an embodiment, the compounds of Formula 1 are useful in the treatment of diabetes mellitus and obesity. In another embodiment, the compounds of Formula 1 are useful in the treatment of obesity.

As used herein, the term “metabolic syndrome” is a recognized clinical term used to describe a condition comprising combinations of Type II diabetes, impaired glucose tolerance, insulin resistance, hypertension, obesity, increased abdominal girth, hypertriglyceridemia, low HDL, hyperuricaemia, hypercoagulability and/or microalbuminemia. Diabestity typically involves a metabolic syndrome (such as insulin resistance syndrome or syndrome X) defined as a clustering of cardiovascular risk factors (abdominal obesity, hyperinsulinemia, atherogenic dyslipidemia, hypertension and hypercoagulability) that together increase the risk of developing coronary heart disease and type 2 diabetes. Metabolic syndrome is a clinical disorder where increased insulin concentration is observed with associated conditions such as visceral obesity, hyperlipidemia, atherogenic dyslipidemia, hyperglycemia, hypertension, hyperurecemia and renal dysfunction.

An ACC-mediated disease or condition also includes fatty liver, hepatic steatosis, hepatitis, non-alcoholic hepatitis, non-alcoholic steatohepatitis (NASH), alcoholic hepatitis, acute fatty liver, fatty liver of pregnancy, drug-induced hepatitis, erythrohepatic protoporphyria, iron overload disorders, hereditary hemochromatosis, hepatic fibrosis, hepatic cirrhosis, hepatoma and conditions related thereto.

An ACC-mediated disease or condition also includes, but is not limited to, a disease or condition which is, or is related to primary hypertriglyceridemia, or hypertriglyceridemia secondary to another disorder or disease, such as hyperlipoproteinemias, familial histiocytic reticulosis, lipoprotein lipase deficiency, apolipoprotein deficiency (such as ApoCII deficiency or ApoE deficiency), and the like, or hypertriglyceridemia of unknown or unspecified etiology.

An ACC-mediated disease or condition also includes a disorder of polyunsaturated fatty acid (PUFA) disorder, or a skin disorder, including, but not limited to, eczema, acne, psoriasis, keloid scar formation or prevention, diseases related to production or secretions from mucous membranes, such as monounsaturated fatty acids, wax esters, and the like.

An ACC-mediated disease or condition also includes inflammation, sinusitis, asthma, pancreatitis, osteoarthritis, rheumatoid arthritis, cystic fibrosis, and pre-menstrual syndrome.

An ACC-mediated disease or condition also includes but is not limited to a disease or condition which is, or is related to cancer, neoplasia, malignancy, metastases, tumours (benign or malignant), carcinogenesis, hepatomas and the like.

An ACC-mediated disease or condition also includes a condition where increasing lean body mass or lean muscle mass is desired, such as is desirable in enhancing performance through muscle building. Myopathies and lipid myopathies such as carnitine palmitoyltransferase deficiency (CPT I or CPT II) are also included herein. Such treatments are useful in humans and in animal husbandry or companion animals, including for administration to canine, feline, bovine, porcine or avian domestic animals or any other animal to reduce triglyceride production or body weight and/or provide leaner meat products and/or healthier animals.

An ACC-mediated disease or condition also includes a disease or condition which is, or is related to, neurological diseases, psychiatric disorders, multiple sclerosis, eye diseases, and immune disorders.

An ACC-mediated disease or condition also includes a disease or condition which is, or is related to, viral diseases or infections including but not limited to all positive strand RNA viruses, coronaviruses, SARS virus, SARS-associated coronavirus, Togaviruses, Picornaviruses, Coxsackievirus, Yellow Fever virus, Flaviviridae, Filoviridae, ALPHAVIRUS (TOGAVIRIDAE) including Rubella virus, Eastern equine encephalitis virus, Western equine encephalitis virus, Venezuelan equine encephalitis virus, Sindbis virus, Semliki forest virus, Chikungunya virus, O′nyong′nyong virus, Ross river virus, Mayaro virus, Alphaviruses; ASTROVIRIDAE including Astrovirus, Human Astroviruses; CALICIVIRIDAE including Vesicular exanthema of swine virus, Norwalk virus, Calicivirus, Bovine calicivirus, Pig calcivirus, Hepatitis E; CORONAVIRIDAE including Coronavirus, SARS virus, Avian infectious bronchitis virus, Bovine coronavirus, Canine coronavirus, Feline infectious peritonitis virus, Human coronavirus 299E, Human coronavirus OC43, Murine hepatitis virus, Porcine epidemic diarrhea virus, Porcine hemagglutinating encephalomyelitis virus, Porcine transmissible gastroenteritis virus, Rat coronavirus, Turkey coronavirus, Rabbit coronavirus, Berne virus, Breda virus; FLAVIVIRIDAE including Hepatitis C virus, West Nile virus, Yellow Fever virus, St. Louis encephalitis virus, Dengue Group, Hepatitis G virus, Japanese B encephalitis virus, Murray Valley encephalitis virus, Central European tick-borne encephalitis virus, Far Eastern tick-borne encephalitis virus, Kyasanur forest virus, Louping ill virus, Powassan virus, Omsk hemorrhagic fever virus, Kumilinge virus, Absetarov anzalova hypr virus, Ilheus virus, Rocio encephalitis virus, Langat virus, Pestivirus, Bovine viral diarrhea, Hog cholera virus, Rio Bravo Group, Tyuleniy Group, Ntaya Group, Uganda S Group, Modoc Group; PICORNAVIRIDAE including Coxsackie A virus, Rhinovirus, Hepatitis A virus, Encephalomyocarditis virus, Mengovirus, ME virus, Human poliovirus 1, Coxsackie B; POTYVIRIDAE including Potyvirus, Rymovirus, Bymovirus. Additionally it can be a disease or infection caused by or linked to Hepatitis viruses, Hepatitis B virus, Hepatitis C virus, human immunodeficiency virus (HIV) and the like. Treatable viral infections include those where the virus employs an RNA intermediate as part of the replicative cycle (hepatitis or HIV); additionally it can be a disease or infection caused by or linked to RNA negative strand viruses such as influenza and parainfluenza viruses.

In one embodiment, the compounds of the inventions are useful in the treatment of elevated levels of lipids, cardiovascular diseases, diabetes, obesity, and metabolic syndrome.

The term “treating” means to relieve, alleviate, delay, reduce, reverse, improve or prevent at least one symptom of a condition in a subject. The term “treating” may also mean to arrest, delay the onset (i.e., the period prior to clinical manifestation of a disease), manage and/or reduce the risk of developing or worsening a condition.

An “effective amount” means the amount of a compound of formula 1 that, when administered to a patient (e.g., a mammal) for treating a disease, is sufficient to effect such treatment for the disease to achieve the objectives of the invention. The “effective amount” will vary depending on the compound, the disease and its severity and the age, weight, etc., of the patient to be treated.

A subject or patient in whom administration of the therapeutic compound is an effective therapeutic regimen for a disease or disorder is preferably a human, but can be any animal, including a laboratory animal in the context of a clinical trial or screening or activity experiment. Thus, as can be readily appreciated by one of ordinary skill in the art, the methods, compounds and compositions of the present invention are particularly suited to administration to any animal, particularly a mammal, and including, but by no means limited to, humans, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., i.e., for veterinary medical use.

In some embodiments, the compounds of the present invention are administered as a mono-therapy. In other embodiments, the compounds of the present invention are administered as part of a combination therapy. For example, a compound of formula I may be used in combination with other drugs or therapies that are used in the treatment/prevention/suppression or amelioration of the diseases or conditions for which compounds of formula I are useful.

Such other drug(s) may be administered, by a route and in an amount commonly used therefore, contemporaneously or sequentially with a compound of formula I. When a compound of formula I is used contemporaneously with one or more other drugs, a pharmaceutical unit dosage form containing such other drugs in addition to the compound of formula I may be employed. Accordingly, the pharmaceutical compositions of the present invention include those that also contain one or more other active ingredients, in addition to a compound of formula I.

### Measurement of ACC Inhibition

The procedures for measuring ACC1 inhibition and ACC2 inhibition are identical except for the source of the enzyme, and is based upon standard procedure as described by Harwood et al. (J. Biol. Chem. 2006; 28:37099-37111), the contents of which are incorporated herein by reference. For measurement of ACC activity and assessment of ACC inhibition, test compounds were dissolved in dimethylsulfoxide (DMSO) in polypropylene tubes. The reaction was set up in the 96-well plates with the respective positioning of control compounds, reference standards and test compounds. 88 μl of the substrate mix carrying 25 μM acetyl CoA (ACA) and 4 mM ATP in a assay buffer (containing 50 mM HEPES, 2 mM MgCL₂, 2 mM DTT, 10 mM Tri-potassium citrate, 12 mM KHCO₃ and 0.75 mg/ml BSA) was added to the respective wells of the assay plate, already carrying 12 μl of the test compound or reference standard in the wells. The min wells meant for the background carried 120 μl of 5N Hydrochloric acid. This was followed by the addition of 10 μl of the radioactive ligand [¹⁴C—NaHCO₃; Amersham Biosciences; specific activity: 56mCi/mmol] to all wells of the assay plate. The reaction was initiated by the addition of 500 ng of citrate-activated enzyme to all the wells. The plate was incubated at 37° C. for 60 minutes. After incubation, the reaction was terminated by adding 120 μl of 5N Hydrochloric acid in all wells except min wells. The plate was transferred to a 70° C. vacuum oven (fitted with an elaborate system of acid trap and charcoal traps) and was kept for overnight. The residue in the dried wells was re-suspended with 30 μl of the distilled water followed by the addition of 200 μl of Microscint20 to each well. The plate was sealed with plate sealer and was kept on a plate shaker with vigorous shaking for 4-5 hours (or till the counts stabilize). Subsequently, counting of the plates was carried out using MicroBeta Trilux (Counting time 30 sec/well).

The IC₅₀ of the compound to inhibit ACC1 and ACC2 activity was determined by the concentration of the compound required to inhibit 50% of the total activity of the enzyme (measured in the absence of any compound/inhibitor). The selectivity of the compounds for ACC2 over ACC1 was determined by dividing IC₅₀ of the compound for ACC1 by IC₅₀ of the compound for ACC2.

The compounds of the present invention typically exhibit potency values of greater than 50% inhibition in the range 20 nM to 5 μM in the ACC inhibition assay.

### EXAMPLES

The present invention will now be further described by way of the following non-limiting examples. In applying the disclosure of these examples, it should be kept clearly in mind that other and different embodiments of the synthetic methods disclosed according to the present invention will no doubt suggest themselves to those of skill in the relevant art.

The entire disclosures of all applications, patents and publications, cited above and below, are hereby incorporated by reference.

In the foregoing and in the following examples, all temperatures are set forth uncorrected in degrees Celsius.

The following abbreviations are used herein: DCM (dichloromethane), DMF (dimethylformamide), EDC.HCl (1-ethyl-3-[3-dimethylaminopropyl]carbodiimide hydrochloride), HOBt (1-hydroxybenzotriazole), TFA (trifluoroacetic acid), THF (tetrahydrofuran), EtOAc (ethyl acetate), MeOH (methanol), Pd(OAc)₂ (palladium acetate), K₂CO₃ (potassium carbonate), Boc (tert-butoxycarbonyl), Na₂SO₄ (sodium sulphate), NaHCO₃ (sodium bicarbonate), HCl (hydrochloric acid), HBr (hydrogen bromide), brine (saturated sodium chloride solution), CDCl₃ (deuterated chloroform), Cs₂CO₃ (cesium carbonate), NaOH (sodium hydroxide), KOH (potassium hydroxide), conc. (concentrated), Celite (diatomaceous earth), NMR (nuclear magnetic resonance), DMSO-d₆ (deuterated dimethyl sulfoxide), LCMS (liquid chromatography mass spectrometry), TEA (triethylamine), AIBN (azobisisobutyronitrile), NBS (N-bromosuccinimide), NCS(N-chlorosuccinimide), ppm (parrts per million chemical shift), POCl₃ (phosphorous oxychloride), HBr (hydrogen bromide), CuI (copper(I) iodide), NaBH₄ (sodium borohydride).

### Starting Compounds and Intermediates

6-Methoxy-3,4-dihydro-2H-isoquinolin-1-one was prepared from 3-methoxyphenethylamine according to the procedure reported in J. Med. Chem. 1987, 30, 2208-2216.

6-methoxy-1,2,3,4-tetrahydroisoquinoline was prepared from 6-Methoxy-3,4-dihydro-2H-isoquinolin-1-one using the procedure reported in J. Med. Chem. 1987, 30, 2208-2216.

6-Methoxy-1,2,3,4-tetrahydroisoquinoline was converted to 1,2,3,4-tetrahydroisoquinolin-6-ol hydrobromic acid salt according to the procedure reported in J. Med. Chem. 1987, 30, 2208-2216. TEA (9.43 ml, 0.0678 mol) was added to a stirred solution of 1,2,3,4-tetrahydroisoquinolin-6-ol hydrobromic acid salt (5.20 g, 0.0226 mol) in DCM (40 ml) at 0° C. and stirred for 10 min at same temperature. Boc₂O (5.32 ml, 0.0249 mol) was added to the reaction mixture at 0° C. After the addition, the reaction was warmed to room temperature and stirred for two hours. The reaction mixture was diluted with DCM, and washed with water and brine. The organic extract was dried over anhydrous sodium sulphate. Evaporation of the solvent generated a residue. Purification of the residue by column chromatography afforded 5.00 g (80.80%) of 6-hydroxy-3,4-dihydro-1H-isoquinoline-2-carboxylic acid tert-butyl ester. LCMS: [M−H]⁻ 248.4; 1H-NMR (CDCl₃): 6.952-6.972 (d, 1H, J=8 Hz), 6.662-6.681 (d, 1H, J=7.6 Hz), 6.618 (s, 1H), 4.489 (s, 2H), 3.612 (bs, 2H), 2.769 (bs, 2H), 1.488 (s, 9H). 6-Hydroxy-3,4-dihydro-1H-isoquinoline-2-carboxylic acid tert-butyl ester was converted to 6-acetyl-3,4-dihydro-1H-isoquinoline-2-carboxylic acid tert-butyl ester using the procedure reported in WO2007/106349. To a solution of 6-acetyl-3,4-dihydro-1H-isoquinoline-2-carboxylic acid tert-butyl ester (1.25 g, 4.54 mmol) in ethyl acetate (20 ml) was added conc. HCl (10 ml) and the reaction mixture was stirred for 8 h at room temperature. The solvent was evaporated to obtain a crude residue which was dissolved in water and basified with NaHCO₃. The aqueous solution was extracted with ethyl acetate twice and the combined extracts dried over anhydrous Na₂SO₄. The solvent was evaporated to obtain a crude residue. Purification of the crude residue by column chromatography (silica gel 60-120 mesh, MeOH:DCM 0.2:9.8) afforded (0.5 g, 62.85%) of 1-(1,2,3,4-tetrahydroisoquinolin-6-yl)ethanone. ¹H-NMR (DMSO-d₆): 7.654 (bs, 2H), 7.124-7.143 (d, 1H, J=7.6 Hz), 3.871 (s, 2H), 2.921-2.948 (t, 2H, J=5.6 Hz), 2.733-2.746 (m, 2H), 2.517 (s, 3H).

6-Acetyl-3,4-dihydro-1H-isoquinoline-2-carboxylic acid tert-butyl ester (600 mg, 2.18 mmol), ammonium acetate (2.0 g, 2.6.mol) and 4 Å molecular sieves powder (2.0 g) were taken together in 25 ml of methanol and stirred for 20 min at room temperature. Sodium cyanoborohydride (205.5 mg, 3.270 mol) was added to the reaction mixture at room temperature and the reaction was refluxed for 12 h. The reaction mixture was cooled to room temperature, the insolubles filtered through Celite and the solvent was evaporated. Water was added to the crude and it was extracted with ethyl acetate twice. The combined organic extracts were washed with water and brine and dried over anhydrous sodium sulphate. The solvent was evaporated under reduced pressure to obtain crude 6-(1-aminoethyl)-3,4-dihydro-1H-isoquinoline-2-carboxylic acid tert-butyl ester which was taken for the next step without any purification. TEA (2.51 ml, 0.018 mol) was added to a stirred solution of 6-(1-aminoethyl)-3,4-dihydro-1H-isoquinoline-2-carboxylic acid tert-butyl ester (3.3 g, 0.0120 mol) in THF (70 ml) at 0° C. over a period of 5 min and stirred further for 15 min at this temperature. Acetyl chloride (0.942 ml, 0.0132 mol) was added to the reaction mixture at 0° C. and the reaction was stirred for further 20 min at 0° C. Water was added to the reaction mixture and the reaction mass extracted with ethyl acetate twice. The combined organic extracts were washed with brine, dried over anhydrous sodium sulphate and evaporated under vacuum to obtain a residue. Purification of the residue by column chromatography (silica gel, methanol/DCM 0.5:9.5) afforded 1.65 g (43.4%) of 6-(1-acetylaminoethyl)-3,4-dihydro-1H-isoquinoline-2-carboxylic acid t-butyl ester. LCMS [M−56]⁻ 263.5, 1H-NMR (CDCl₃): 7.123-7.142 (d, 1H, J=7.6 Hz), 7.083 (bs, 2H), 5.603-5.619 (d, 1H, J=6.4 Hz), 5.062-5.097 (m, 1H), 4.546 (s, 2H), 3.633 (bs, 2H), 2.806-2.834 (t, 2H, J=5.6 Hz), 1.981 (s, 3H), 1.462-1,484 (m, 12H). Trifluoroacetic acid (1.932 ml, 0.026 mol) was added to a stirred solution of 6-(1-acetylaminoethyl)-3,4-dihydro-1H-isoquinoline-2-carboxylic acid tert-butyl ester (1.65 g, 0.0052 mol) in DCM (30 ml) at 0° C. over a period of 5 min and the reaction was stirred for 1 h at room temperature. Evaporation of the solvent under vacuum followed by washing and decanting with diethyl ether afforded 1.6 g (93. %) of N-[1-(1,2,3,4-tetrahydroisoquinolin-6-yl)ethyl]acetamide trifluoroacetic acid salt. LCMS [M+H]⁺ 219.4; ¹H-NMR (DMSO-d₆): 8.95 (bs, 2H), 8.219-8.238 (d, 1H, J=7.6 Hz), 7.137-7.143 (d, 2H, J=2.4 Hz), 7.110 (s, 1H), 4.806-4.844 (m, 1H), 4.203-4.226 (t, 2H, J=4.4 Hz), 3.343-3.396 (m, 2H), 2.938-2.969 (t, 2H, J=6.0 Hz), 1.806 (s, 3H), 1.278-1.296 (d, 3H, J=7.2 Hz).

4-Hydroxybenzaldehyde (3.00 g, 0.0246 mol), bromomethylcyclopropane (3.58 ml, 0.0369 mol) and K₂CO₃ (6.8 g, 0.050 mol) were taken in acetone (80 ml) and refluxed for 24 h. The reaction mixture was cooled to room temperature, the insolubles filtered through Celite and the solvent was evaporated to obtain a crude residue. Purification of the residue by column chromatography (silica gel 60-120 mesh, EtOAc:Hexane 0.5:9.5) afforded 3.6 g (83.14%) of 4-cyclopropylmethoxybenzaldehyde. LCMS [M+H]⁺ 177.0 1H-NMR (CDCl₃): 9.877 (s, 1H), 7.813-7.833 (d, 2H, J=8 Hz), 6.984-7.004 (d, 2H, J=8 Hz), 3.882-3.899 (d, 2H, J=6.8 Hz), 1.259-1.295 (m, 1H), 0.669-0.687 (m, 2H), 0.383 (bs, 2H). NaBH₄ (0.727 g, 19.2 mmol) was added to a stirred solution of 4-cyclopropylmethoxybenzaldehyde (2.7 g, 15.3 mmol) in methanol (50 ml) at 0° C. over a period of 15 min and the reaction mixture was stirred for 30 min at room temperature. The solvent was evaporated to obtain a residue which was dissolved in water and extracted with ethyl acetate twice. The combined organic extracts were washed with water and brine and dried over anhydrous Na₂SO₄. The ethyl acetate layer was evaporated to obtain 2.2 g (83.65%) of 4-cyclopropylmethoxyphenyl)methanol. 1H-NMR (CDCl₃): 7.268-7.289 (d, 2H, J=8.4 Hz), 6.884-6.905 (d, 2H, J=8.4 Hz), 4.607-4.621 (d, 2H, J=5.6 Hz), 3.796-3.813 (d, 2H, J=6.8 Hz), 1.592-1.561 (m, 1H), 1.262-1.290 (m, 1H), 0.636-0.655 (d, 2H, J=7.6 Hz), 0.346-0.357 (d, 2H, J=4.4 Hz). Aqueous HBr (3 ml) was added to a stirred solution of 4-cyclopropylmethoxyphenyl)methanol (300 mg, 1.683 mmol) in ether (20 ml) at 0° C. and the reaction mixture stirred for 30 min at 0° C. The solvent was evaporated to obtain crude which was dissolved in water and extracted with ether thrice. The total organic extracts were washed with water, dried over Na₂SO₄ and concentrated under vacuum to obtain (260 mg, 64%) of 1-bromomethyl-4-cyclopropylmethoxybenzene. 1H-NMR (CDCl₃): 7.293-7.313 (d, 2H, J=8.0 Hz), 6.846-6.866 (d, 2H, J=8.0 Hz), 4.496 (s, 2H), 3.787-3.804 (d, 2H, J=6.8 Hz), 1.262 (bs, 1H), 0.635-0.651 (d, 2H, J=6.4 Hz), 0.348 (bs, 2H)

Cyclopropyl methyl bromide (1.4 ml, 14.46 mmol), sodium iodide (0.986 g, 6.6 mmol) and potassium carbonate (3.64 g, 26.3 mmol) were added sequentially to a stirred solution of 4-hydroxybenzoic acid methyl ester (2.0 g, 13.14 mmol), in 25 ml of acetone at room temperature and the mixture was refluxed for 44 h. The solvent was evaporated to obtain a residue to which 10% NaOH (80 ml) was added. The solution was extracted with DCM twice. The combined DCM layers were washed with water and brine and dried over anhydrous Na₂SO₄. The solvent was evaporated to obtain 2.5 g (92.25%) of 4-cyclopropylmethoxybenzoic acid methyl ester. LCMS: [M+H]⁺ 270.3; 1H-NMR (CDCl₃): 7.964-7.986 (d, 2H, J=8.8 Hz), 6.895-6.917 (d, 2H, J=8.8 Hz), 3.879 (s, 3H), 3.849-3.867 (d, 2H, J=7.2 Hz), 1.262-1.281 (m, 1H), 0.639-0.671 (m, 2H), 0.360-0.372 (m, 2H). 2N NaOH (7 ml) was added to a stirred solution of 4-cyclopropylmethoxy benzoic acid methyl ester (1.5 g, 7.3 mmol) in methanol (15 ml) and the reaction mixture was stirred for 2 h at 60° C. The solvent was evaporated and the remaining residue was dissolved in water. The of the solution was adjusted to pH 2-2.5 with 2N HCl solution at 0° C. and the precipitated solid was filtered and washed with water. The solid compound was re-dissolved in ethyl acetate and washed with brine before drying over anhydrous Na₂SO₄. The solvent was evaporated to obtain 1.35 g (96.56%) of 4-cyclopropylmethoxybenzoic acid. 1H-NMR (DMSO): 12.513 (s, 1H), 7.838-7.860 (d,2H, J=8.8 Hz), 6.965-6.986 (d, 2H, J=8.4 Hz), 3.863-3.880 (d, 2H, J=6.8 Hz), 1.216 (bs, 1H), 0.552-0.570 (m, 2H), 0.313-0.324 (m, 2H).

6-(1-Aminoethyl)-3,4-dihydro-1H-isoquinoline-2-carboxylic acid tert-butyl ester was reacted with methyl chloroformate to afford 6-(1-methoxycarbonylaminoethyl)-3,4-dihydro-1H-isoquinoline-2-carboxylic acid tert-butyl ester in 70% yield. LCMS: [M−56]⁻ 279.4. 6-(1-Methoxycarbonylaminoethyl)-3,4-dihydro-1H-isoquinoline-2-carboxylic acid tert-butyl ester was reacted with TFA to afford [1-(1,2,3,4-tetrahydroisoquinolin-6-yl)ethyl]carbamic acid methyl ester trifluoroacetic acid salt in 30% yield. LCMS: [M+H]⁺ 235.4.

N-chlorosuccinimide (1.1 g, 8.18 mmol) was added to a solution of 4-hydroxybenzaldehyde (1.0 g, 8.18 mmol) in 10 ml of chloroform and the mixture was heated at 50° C. for 15 h. The reaction mixture was cooled and the solvent was evaporated. The residue was dissolved in ethyl acetate (25 ml), washed with water and brine, and dried over anhydrous sodium sulphate. Evaporation rendered crude material which was purified by silica column and was eluted with 12% ethyl acetate in hexane to yield 1.1 g (86%) 3-chloro-4-hydroxybenzaldehyde. 1H-NMR (400 MHz, CDCl₃) δ (ppm): 9.840 (s, 1H), 7.898-7.894 (d, 1H, J=1.6), 7.749-7.724 (dd, 1H, J=8.4 Hz), 7.164-7.143 (d, 1H, J=8.4 Hz), 6.288 (s, 1H).

3-Chloro-4-hydroxybenzaldehyde was reacted with n-propyl iodide to afford 47% of 3-chloro-4-isopropoxybenzaldehyde. ¹H-NMR (400 MHz, CDCl₃) δ (ppm): 9.845 (s, 1H), 7.907-7.901 (d, 1H, J=2.4), 7.759-7.734 (dd, 1H, J=8.8, 1.6 Hz), 7.026-7.005 (d, 1H, J=8.4), 4.105-4.073 (t, 2H), 1.936-1.884 (m, 2H), 1.096-1.057 (t, 3H). 3-Chloro-4-propoxybenzaldehyde was converted to (3-chloro-4-propoxyphenyl)methanol in 50% yield. 1H-NMR (400 MHz, CDCl₃) δ (ppm): 7.377-7.382 (d, 1H, J=2 Hz), 7.173-7.198 (dd, 1H, J=10 Hz, 1.6 Hz), 6.906 (s, 1H), 4.597 (s, 2H), 3.975-4.008 (t, 2H, J=6.4 Hz), 1.814-1.884 (m, 2H), 1.034-1.067 (t, 3H, J=5.6 Hz). (3-Chloro-4-propoxyphenyl)methanol was converted to 4-bromomethyl-2-chloro-1-propoxybenzene in 50% yield. 1H-NMR (CDCl₃), δ (ppm): 7.408 (s, 1H), 7.213-7.234 (d, 1H, J=8.4 Hz), 6.851-6.872 (d, 1H, J=8.4 Hz), 4.434 (s, 2H), 3.977-4.009 (t, 2H, J=6.4 Hz), 1.833-1.886 (m, 2H), 1.049-1.085 (t, 3H, J=6.8 Hz). 3-Chloro-4-hydroxybenzaldehyde was reacted with isopropyl iodide to afford 57% of 3-chloro-4-isopropoxybenzaldehyde. ¹H-NMR (400 MHz, CDCl₃) δ (ppm): 9.835 (s, 1H), 7.901-7.896 (d, 1H, J=2 Hz), 7.747-7.720 (dd, 1H, J=10.8, 1.6 Hz), 7.034-7.013 (d, 1H, J=8.4 Hz), 4.739-4.678 (m, 1H), 1.439-1.424 (d, 6H, J=6 Hz). 3-Chloro-4-isopropoxybenzaldehyde was reacted with sodium borohydride to afford 0.8 g (99%) of (3-chloro-4-isopropoxyphenyl)methanol. ¹H-NMR (400 MHz, CDCL3) δ (ppm): 7.382 (s, 1H), 7.191-7.170 (d, 1H, J=8.4 Hz), 6.941-6.920 (d, 1H, J=8.4 Hz), 4.597 (s, 2H), 4.571-4.510 (m, 1H), 1.382-1.366 (d, 6H, J=6 Hz). (3-Chloro-4-isopropoxyphenyl)methanol was converted to 4-bromomethyl-2-chloro-1-isopropoxybenzene in 45% yield. 1H-NMR (CDCl₃) δ (ppm): 7.406 (s, 1H), 7.201-7.222 (d, 1H, J=8.4 Hz), 6.875-6.896 (d, 1H, J=8.4 Hz), 4.524-4.584 (m, 1H), 4.427 (s, 2H), 1.369-1.385 (d, 6H, J=6.4 Hz).

Isobutyl bromide (1.58 g, 11.5 mmol) was added to a mixture of 3-chloro-4-hydroxybenzaldehyde (1.5 g, 9.6 mmol) and potassium carbonate (2.66 g, 19.2 mmol) in 15 ml of N,N-dimethylformamide. The reaction mixture was heated at 75° C. for 12 h. The reaction mixture was cooled to room temperature, diluted with water and extracted with ethyl acetate. The combined organic layer was washed with water, brine, dried over anhydrous sodium sulphate and evaporated to render 1.8 g (85%) of 3-chloro-4-isobutoxybenzaldehyde which was taken to the next step without any purification. 3-Chloro-4-isobutoxybenzaldehyde was reacted with sodium borohydride to afford 94% of (3-chloro-4-isobutoxyphenyl)methanol. ¹H-NMR (400 MHz, CDCl₃), δ (ppm): 7.376 (s, 1H), 7.191-7.171 (d, 1H, J=8), 6.893-6.872 (d, 1H, J=8.4), 4.593 (s, 2H), 3.791-3.775 (d, 2H, J=6.4), 2.181-2.080 (m, 1H), 1.066-1.050 (d, 6H, J=6.4). (3-Chloro-4-isobutoxyphenyl)methanol was converted in 56% yield to 4-bromomethyl-2-chloro-1-isobutoxybenzene. ¹H-NMR (CDCl₃), δ (ppm) 7.406 (s, 1H), 7.207-7.227 (d, 1H, J=8 Hz), 6.837-6.858 (d, 1H, J=8.4 Hz), 4.433 (s, 2H), 3.774-3.790 (d, 2H, J=6.4 Hz), 2.131-2.164 (m, 1H), 1.046-1.063 (d, 6H, J=6.8 Hz).

3-Chloro-4-hydroxybenzaldehyde was reacted with bromomethylcyclopropane in 97% yield to furnish 3-chloro-4-cyclopropylmethoxybenzaldehyde. ¹H-NMR (400 MHz, CDCl₃) δ (ppm): 9.845 (s, 1H), 7.909 (s, 1H), 7.749-7.728 (d, 1H, J=8.4 Hz), 7.011-6.990 (d, 1H, J=8.4 Hz), 4.001-3.985 (d, 2H, J=6.4 Hz), 1.345 (m, 1H), 0.705-0.686 (d, 2H, J=7.6 Hz), 0.434-0.424 (d, 2H, J=4 Hz). 3-Chloro-4-cyclopropylmethoxybenzaldehyde was reacted with sodium borohydride to afford quantitatively (3-chloro-4-cyclopropylmethoxyphenyl)methanol. ¹H-NMR (400 MHz, CDCl₃) δ (ppm): 7.380 (s, 1H), 7.186-7.166 (d, 1H, J=8 Hz), 6.904-6.883 (d, 1H, J=8.4 Hz), 4.597 (s, 2H), 3.894-3.878 (d, 2H, J=6.4 Hz), 1.308-1.238 (m, 1H), 0.653-0.635 (d, 2H, J=7.2 Hz), 0.388-0.377 (d, 2H, J=4.4 Hz). (3-Chloro-4-cyclopropylmethoxyphenyl)methanol was reacted with aqueous hydrobromic acid (10 ml) to afford 4-bromomethyl-2-chloro-1-cyclopropylmethoxybenzene. This crude material was taken as such for next step without any purification.

3-Chloro-4-hydroxybenzaldehyde was reacted with ethyl to afford 68% of 3-chloro-4-ethoxybenzaldehyde. ¹H-NMR (400 MHz, CDCl₃) δ (ppm) 9.845 (s, 1H), 7.907-7.902 (d, 1H, J=2), 7.761-7.735 (dd, 1H, J=10.4, 2.0 Hz), 7.028-7.006 (d, 1H, J=8.8), 4.234-4.182 (q, 2H, J=6.8 Hz), 1.535-1.501 (t, 3H, J=6.8 Hz). 3-Chloro-4-ethoxybenzaldehyde was converted quantitatively to (3-chloro-4-ethoxyphenyl)methanol. ¹H-NMR (400 MHz, CDCl₃) δ (ppm): 7.379 (s, 1H), 7.199-7.178 (d, 1H J=8.4 Hz), 6.908-6.887 (d, 1H, J=8.4), 4.6 (s, 2H), 4.134-4.081 (q, 2H, 7.2 Hz), 1.463-1.428 (t, 3H, 6.8 Hz). (3-Chloro-4-ethoxyphenyl)methanol was reacted with aqueous hydrobromic acid. The crude material was taken as such for next step without any purification.

A solution of bromine (0.42 ml, 8.18 mmol) in chloroform (10 ml) was added drop-wise to a solution of 4-hydroxybenzaldehyde (1.0 g, 8.18 mmol) in chloroform (25 ml) at 40° C. The reaction mixture was maintained at 40° C. for two hours. Then the reaction mixture was cooled to room temperature and the solvent was evaporated. The residue was dissolved in ethyl acetate, washed with water, brine, dried over anhydrous sodium sulphate and evaporated to get a residue. Purification of the residue by column chromatography (silica gel 230-400 mesh, EtOAc:hexane 1.2:8.8) afforded 1.2 g (73%) of 3-bromo-4-hydroxybenzaldehyde. ¹H-NMR (400 MHz, CDCl₃) δ (ppm): 9.824 (s, 1H), 8.035 (s, 1H), 7.778-7.757 (d, 1H, J=8.4 Hz), 7.154-7.133 (d, 1H, J=8.4 Hz), 6.632 (s, 1H).

Isopropyl iodide (1.22 g, 7.16 mmol) was added to a mixture of 3-bromo-4-hydroxybenzaldehyde (1.2 g, 5.97 mmol) and potassium carbonate (1.65 g, 11.94 mmol) in DMF (12 ml). The reaction mixture was stirred at room temperature for 2 h. The reaction mixture was diluted with water and extracted with ethyl acetate. The combined organic layers were washed with water, brine, dried over anhydrous sodium sulphate and evaporated to get 0.8 g (55%) of 3-bromo-4-isopropoxybenzaldehyde. ¹H-NMR (400 MHz, CDCl₃) δ (ppm): 9.827 (s, 1H), 8.079 (s, 1H), 7.793-7.772 (d, 1H, J=8.4 Hz), 6.998-6.977 (d, 1H, J=8.4 Hz), 4.740-4.680 (m, 1H), 1.388-1.373 (d, 6H, J=6 Hz). 3-Bromo-4-isopropoxybenzaldehyde was reacted with sodium to quantitatively afford (3-bromo-4-isopropoxyphenyl)methanol. ¹H-NMR (400 MHz, CDCl₃) δ (ppm): 7.558-7.554 (d, 1H, J=1.6), 7.239-7.214 (dd, 1H, J=10, 1.6 Hz), 6.909-6.888 (d, 1H, J=8.4 Hz), 4.596 (s, 2H), 4.573-4.497 (m, 1H), 1.384-1.370 (d, 6H, J=5.6 Hz). (3-Bromo-4-isopropoxyphenyl)methanol was reacted with aqueous hydrobromic acid to afford 2-bromo-4-bromomethyl-1-isopropoxybenzene which was taken for next step without any purification.

3-Bromo-4-hydroxybenzaldehyde was reacted with n-propyl to afford 97% of 3-bromo-4-propoxybenzaldehyde. LC/MS [M+H]³⁰ 243.3; ¹H-NMR (400 MHz, DMSO₆) δ (ppm): 9.83 (s, 1H), 8.07 (s, 1H), 7.87-7.90 (dd, 1H, J=8.6 Hz), 7.27-7.29 (d, 1H, J=8.4 Hz), 4.11-4.14 (tr, 2H, J=6.4 Hz), 1.72-1.79 (m, 2H), 0.98-1.02 (tr, 3H, J=7.2 Hz). 3-Bromo-4-propoxybenzaldehyde was reacted with sodium borohydride to afford 84% of (3-bromo-4-propoxyphenyl)methanol as a yellow solid. ¹H-NMR (400 MHz, DMSO₆) δ (ppm): 7.48 (s, 1H), 7.21-7.23 (dd, 1H, J=8.4 Hz), 7.01-7.03 (d, 1H, J=8.4 Hz), 5.11-5.14 (t, 1H, J=5.8 Hz). 4.38-4.40 (d, 1H, J=5.6 Hz) 3.95-3.98 (m, 2H), 1.69-1.74 (m, 2H), 0.96-1.00 (t, 3H, J=7.6 Hz). (3-Bromo-4-propoxyphenyl)methanol was reacted with aqueous hydrobromic acid to afford crude 2-bromo-4-bromomethyl-1-propoxybenzene which was taken to the next step without any purification.

3-Bromo-4-hydroxybenzaldehyde was reacted with ethyl iodide to afford 94% of 3-bromo-4-ethoxybenzaldehyde. LC/MS [M+H]⁺ 229.3; ¹H-NMR (400 MHz, DMSO₆) δ (ppm): 9.83 (s, 1H), 8.07 (s, 1H), 7.88-7.90 (d, 1H, J=8.6 Hz), 7.27-7.29 (d, 1H, J=8.4 Hz), 4.20-4.25 (m, 2H), 1.35-1.39 (tr, 3H, J=7 Hz). 3-Bromo-4-ethoxybenzaldehyde was reacted with sodium to afford 90% of (3-bromo-4-propoxyphenyl)methanol. ¹H-NMR (400 MHz, DMSO₆) δ (ppm): 7.48 (s, 1H), 7.21-7.23 (d, 1H, J=8.4 Hz), 7.01-7.03 (d, 1H, J=8.4 Hz), 5.11-5.14 (tr, 1H, J=5.8 Hz). 4.33-4.40 (m, 2H), 3.96-4.09 (m, 2H), 1.35-1.39 (tr, 3H, J=7 Hz). (3-Bromo-4-ethoxyphenyl)methanol was reacted with aq. HBr to afford crude 2-bromo-4-bromomethyl-1-ethoxybenzene which was taken to the next step without any purification.

3-Bromo-4-hydroxybenzaldehyde was reacted with bromomethylcyclopropane to afford 74% of 3-bromo-4-cyclopropylmethoxy benzaldehyde. ¹H-NMR (400 MHz, DMSO₆) δ (ppm): 9.82 (s, 1H), 8.07 (s, 1H), 7.86-7.88 (d, 1H, J=8 Hz), 7.25-7.27 (d, 1H, J=8.4 Hz), 4.03-4.05 (d, 2H, J=7.2 Hz), 1.21-1.26 (m, 1H), 0.56-0.61 (m, 2H), 0.35-0.39 (m, 2H). 3-Bromo-4-cyclopropylmethoxybenzaldehyde was reacted with sodium borohydride to afford 71% of (3-bromo-4-cyclopropylmethoxyphenyl)methanol. ¹H-NMR (400 MHz, DMSO₆) δ (ppm): 7.48 (s, 1H), 7.19-7.22 (d, 1H, J=8.4 Hz), 6.99-7.01 (d, 1H, J=8.4 Hz), 5.11-5.14 (tr, 1H, J=5.8 Hz). 4.38-4.40 (d, 2H J=5.6 Hz,), 3.86-3.88 (d, 2H, J=6.4 Hz), 1.19-1.22 (m, 1H), 0.52-0.57 (m, 2H), 0.31-0.34 (m, 2H). (3-Bromo-4-cyclopropylethoxyphenyl)methanol was reacted with aq. HBr to afford crude 2-bromo-4-bromomethyl-1-cyclopropylethoxybenzene which was taken to the next step without any purification.

3-Bromo-4-hydroxybenzaldehyde was reacted with isobutyl bromide to afford 74% of 3-bromo-4-isobutyloxy benzaldehyde. LC/MS [M+H]⁺ 257.3; ¹H-NMR (400 MHz, DMSO-d₆) δ (ppm): 9.83 (s, 1H), 8.07 (s, 1H), 7.87-7.89 (d, 1H, J=8 Hz), 7.26-7.28 (d, 1H, J=8.4 Hz), 3.94-3.95 (d, 2H, J=6.4 Hz), 2.05-2.08 (m, 1H), 0.96-0.99 (d, 6H, J=14 Hz). 3-Bromo-4-isobutoxybenzaldehyde was reacted with sodium borohydride to afford 71% of (3-bromo-4-isobutoxyphenyl)methanol. ¹H-NMR (400 MHz, DMSO₆) δ (ppm): 7.48 (s, 1H), 7.21-7.23 (d, 1H, J=8.4 Hz), 7.00-7.02 (d, 1H, J=8.4 Hz), 5.11-5.14 (t, 1H, J=5.8 Hz). 4.38-4.40 (d, 2H J=5.6 Hz,), 3.77-3.79 (d, 2H, J=6 Hz), 1.97-2.05 (m, 1H), 0.94-0.98 (d, 6H, J=14 Hz). (3-Bromo-4-isobutoxyphenyl)methanol and aq. HBr were reacted to afford crude 2-bromo-4-bromomethyl-1-isobutoxybenzene which was taken to the next step without any purification.

m-cresol (10 g, 92.5 mmol) and isopropyl bromide (10.5 ml, 111 mmol) were taken in ethanol and heated to reflux. While at reflux, KOH (7.79 g, 138.7 mmol) dissolved in water (10 ml) was added drop-wise and the reaction mixture was refluxed for another 4 h. Ethanol was evaporated and the reaction mass was diluted with water. The aqueous solution was extracted with ethyl acetate, washed with cold water and brine before drying over anhydrous sodium sulphate to obtain 87% of 1-isopropoxy-3-methylbenzene. LC/MS [M+H]⁺ 151.1. 1-Isopropoxy-3-methylbenzene (2 g, 13.3 mmol) was added drop-wise to a mixture of POCl₃ (1.2 ml, 13.3 mmol) and DMF (3.7 ml, 48.2 mmol) at 0° C. and the resultant mixture was heated at 80-90° C. for 5 h. The reaction mixture was then poured onto crushed ice. The solution was neutralized using sodium acetate after which it was extracted with DCM and dried over anhydrous Na₂SO₄. The organic layer was evaporated under vacuum to obtain a crude residue. Purification of crude by column chromatography (silica gel 60-120 mesh, EtOAc:hexane 0.2:9.8) afforded (600 mg, 26%) of 4-isopropoxy-2-methylbenzaldehyde as a colorless oil. ¹H-NMR (400 MHz, CDCl₃) δ (ppm):7.718-7.740(d, 1H J=8.8 Hz) 6.794-6.821 (d, 1H, J=8.4 Hz), 6.711-6.715 (d, 1H, J=1.6 Hz), 4.608-4.7 (m, 1H), 2.633 (s, 3H), 1.334-1.358 (d, 6H, J=9.6 Hz). 4-Isopropoxy-2-methylbenzaldehyde was reacted with sodium borohydride to afford 84% of (4-isopropoxy-2-methylphenyl)methanol. ¹H-NMR (400 MHz, CDCl₃) δ (ppm): 7.199-7.219 (d, 1H, J=8 Hz), 6.693-6.734 (t, 2H, J=8 Hz), 4.628 (s, 2H), 4.506-4.564 (m, 1H), 2.353 (s, 3H), 1.319-1.377 (d, 6H, J=4.4 Hz). (4-Isopropoxy-2-methylphenyl)methanol and aq. HBr were reacted to afford crude 1-bromomethyl-4-isopropoxy-2-methylbenzene which was taken to the next step without any purification.

1-Ethoxy-3-methylbenzene was converted to 4-ethoxy-2-methylbenzaldehyde in 35% yield. ¹H-NMR (400 MHz, CDCl₃) δ (ppm): 10.106 (s, 1H), 7.726-7.747 (d, 1H, J=8.4 Hz), 6.834-6.808 (dd, 1H, J=10.4, 1.6 Hz), 6.730 (s, 1H), 4.074-4.126 (q, 2H, J=7.2 Hz), 2.639 (s, 3H), 1.418-1.454 (t, 3H, J=7.2 Hz). 4-Ethoxy-2-methylbenzaldehyde was reacted with sodium borohydride to afford 86% of (4-ethoxy-2-methylphenyl)methanol. ¹H-NMR (400 MHz, CDCl₃) δ (ppm): 7.223-7.203 (d, 1H, J=8 Hz), 6.738 (s, 1H), 6.715-6.694 (d, 1H, J=8.4 Hz), 4.629 (s, 2H), 4.046-3.995 (q, 2H), 2.356 (s, 3H), 1.417-1.383 (t, 3H). (4-Ethoxy-2-methylphenyl)methanol (0.2 g, 1.2 mmol) was reacted aqueous hydrobromic acid (10 ml) to afford crude 1-bromomethyl-4-ethoxy-2-methylbenzene which was taken to the next step without any purification.

1-Methyl-3-propoxybenzene was converted in 15% yield to 2-methyl-4-propoxybenzaldehyde. ¹H-NMR (400 MHz, CDCl₃) δ (ppm): 10.108 (s, 1H), 7.727-7,748 (d, 1H, J=8.4 Hz), 6.814-6.842 (dd, 1H, J=11.2 Hz, J=2.4 Hz), 6.735-6.739 (d, 1H, J=1.6 Hz), 3.972-4.0 (t, 2H, J=6.4 Hz), 2.641 (s, 3H), 1.789-1.874(m, 2H), 1.030-1.067 (t, 3H, J=7.6 Hz). 2-Methyl-4-propoxybenzaldehyde was reacted with sodium borohydride to obtain (2-methyl-4-propoxyphenyl)methanol in 80% yield. ¹H-NMR (400 MHz, CDCl₃) δ (ppm): 7.202-7.223 (d, 1H, J=8 Hz), 6.70-6.746 (t, 2H, J=8.4 Hz), 4.626 (s, 2H), 3.892-3.925 (t, 2H, J=6.8 Hz), 2.357 (s, 3H), 1.769-1.840(m, 2H), 1.011-1.047 (t, 2H, J=7.6 Hz). (2-Methyl-4-propoxyphenyl)methanol was converted to 1-bromomethyl-2-methyl-4-propoxybenzene and used in the next step without any purification.

1-Cyclopropylmethoxy-3-methylbenzene was converted to 4-cyclopropylmethoxy-2-methylbenzaldehyde. ¹H-NMR (400 MHz, CDCl₃) δ (ppm): 10.108 (s, 1H), 7.727-7.748 (d, 1H, J=8.4 Hz), 6.821-6.842 (d, 1H, J=8.4 Hz), 6.745 (s, 1H), 3.865-3.882 (d, 2H, J=6.8 Hz), 2.640 (s, 3H), 1.264-1.332 (m, 1H), 0.661-0.680 (d, 2H, J=7.6 Hz), 0.363-0.375 (d, 2H, J=4.8 Hz). 4-Cyclopropylmethoxy-2-methylbenzaldehyde was converted to (4-cyclopropylmethoxy-2-methylphenyl)methanol in 85% yield. ¹H-NMR (400 MHz, CDCl₃) δ (ppm): 7.2047.225 (d, 2H, J=8.4H), 6.703-6.757 (t, 2H, J=8.4 Hz), 4.623-4.635 (d, 2H, J=4.8 Hz), 3.783-3.799 (d, 2H, J=6.4 Hz), 3.799 (d, 2H, J=6.4 Hz), 2.357 (s, 3H), 1.360 (s, 1H), 1.261 (s, 1H), 0.626-0.646 (d, 2H, J=8 Hz), 0.335-0.347 (d, 2H, J=4.8 Hz). (4-Cyclopropylmethoxy-2-methylphenyl)methanol was converted to crude 1-bromomethyl-4-cyclopropylmethoxy-2-methylbenzene.

1-Isobutoxy-3-methylbenzene was converted to 4-isobutoxy-2-methylbenzaldehyde. ¹H-NMR (400 MHz, CDCl₃) δ (ppm): 10.110 (s, 1H), 7.727-7.748 (d, 1H, J=8.4 Hz), 6.821-6.837 (d, 1H, J=6.4 Hz), 6.738 (s, 1H), 3.785-3.795 (d, 2H, J=4 Hz), 2.593 (s, 3H), 2.055-2.138 (m, 1H), 1.045-1.071 (d, 6H, J=10.4 Hz). 4-Isobutoxy-2-methylbenzaldehyde was converted to (4-Isobutoxy-2-methylphenyl)methanol in 87% yield. ¹H-NMR (400 MHz, CDCl₃) δ (ppm): 7.202-7.223 (d1H, J=8.4 Hz), 6.700-6.750 (t, 3H, J=8.4 Hz), 4.629 (s, 2H), 3.700-3.717 (d, 2H, J=6.8 Hz), 2.361 (s, 3H), 2.038-2.104 (m, 1H), 1.010-1.026 (d, 6H, J=6.4 Hz). (4-Isobutoxy-2-methylphenyl)methanol was converted to crude 1-bromomethyl-4-cyclopropylmethoxy-2-methylbenzene.

3-Bromo-4-methylphenol was reacted with isopropyl iodide to afford 98% of 2-bromo-4-isopropoxy-1-methylbenzene. ¹H-NMR (400 MHz, CDCl₃) δ (ppm): 7.106-7.085 (m, 2H), 6.751-6.726 (dd, 1H, J=10, 1.6 Hz), 4.483-4.452 (m, 1H), 2.312 (s, 3H), 1.317-1.302 (d, 6H, J=6 Hz). A mixture of 2-bromo-4-isopropoxy-1-methylbenzene (150 mg, 0.655 mmol), N-bromosuccinimide (116 mg, 0.655 mmol), AIBN (10.9 mg, 0.066 mmol) in carbon tetrachloride (2 ml) was heated at 80° C. for 3 h. The reaction mixture was cooled to room temperature and the solvent was evaporated. The residue was taken up in ethyl acetate, washed with water and brine, dried over anhydrous sodium sulphate and evaporated to afford 200 mg (quant.) of 2-bromo-1-bromomethyl-4-isopropoxybenzene. This crude material was taken for next step without any purification.

3-Chloro-4-methylphenol was reacted with isopropyl iodide to afford 92% of 2-chloro-4-isopropoxy-1-methylbenzene. ¹H-NMR (400 MHz, CDCl₃) δ (ppm): 7.099-7.078 (d, 1H, J=8.4 Hz), 6.898 (s, 1H), 6.705-6.689 (d, 1H, J=6.4 Hz), 4.504-4.444 (m, 1H), 2.289 (s, 3H), 1.323-1.308 (d, 6H, J=6 Hz). 2-Chloro-4-isopropoxy-1-methylbenzene was reacted with N-bromosuccinimide to afford crude 1-bromomethyl-2-chloro-4-isopropoxybenzene which was taken to the next step without any purification.

p-Toluene sulphonylchloride (22.86 g, 11.99 mmol) was added portion wise over a period of 20 minutes to a solution of 2,2,2-trifluoroethanol (10 g, 9.96 mmol) and triethylamine (27.8 ml, 19.99 mmol) in 100 ml of dichloromethane at 0° C. The reaction mixture was stirred at room temperature for 4 hours and diluted with dichloromethane, washed with water, brine, dried over anhydrous sodium sulphate and evaporated. The crude material was washed with hexane to afford Toluene-4-sulfonic acid 2,2,2-trifluoro-ethyl ester in 59% yield. ¹H-NMR (400 MHz, CDCl₃) δ (ppm): 7.807-7.828 (d, 2H, J=8.4 Hz), 7.375-7.396 (d, 2H, J=8.4 Hz), 4.320-4.380 (q, 2H, J=8 Hz), 2.473 (s, 3H).

A mixture of 3-Chloro-4-methylphenol (1 g, 7.0 mmol), Toluene-4-sulfonic acid 2,2,2-trifluoro-ethyl ester (intermediate DII) (1.62 g, 7.7 mmol), potassium carbonate (1.94 g, 14.0 mmol) in 10 ml of N,N-dimethylformamide were heated at 120° C. for 15 hour. Then the reaction mixture was cooled to room temperature, diluted with water and extracted with ethyl acetate. The combined organic extracts were washed with water, brine, dried over anhydrous sodium sulphate and evaporated. The crude material was purified by column over silica to afford 2-Chloro-1-methyl-4-(2,2,2-trifluoro-ethoxy)-benzene. ¹H-NMR (400 MHz, CDCl₃) δ (ppm): 7.144-7.165 (d, 1H, J=8.4 Hz), 6.963 (s, 1H), 6.753-6.780 (dd, 1H, J=10.8 Hz, 2.4 Hz), 4.282-4.343 (q, 2H, J=8.4 Hz), 2.315 (s, 3H).

2-Chloro-1-methyl-4-(2,2,2-trifluoro-ethoxy)-benzene was reacted with N-bromo succinimide to afford crude 1-Bromomethyl-2-chloro-4-(2,2,2-trifluoro-ethoxy)-benzene which was taken to next step without any purification.

4-Methyl-3-nitrophenol was reacted Toluene-4-sulfonic acid 2,2,2-trifluoro-ethyl ester (intermediate DII) similarly as mentioned for intermediate DIII to afford 1-Methyl-2-nitro-4-(2,2,2-trifluoro-ethoxy)-benzene in 72% yield. ¹H-NMR (400 MHz, CDCl₃) δ (ppm): 7.567 (s, 1H), 7.288-7.309 (d, 1H, J=8.4 Hz), 7.122-7.151 (dd, 1H, J=11.6 Hz, 2.8 Hz), 4.368-4.428 (q, 2H, J=8 Hz), 2.557 (s, 3H).

1-Methyl-2-nitro-4-(2,2,2-trifluoro-ethoxy)-benzene was reacted with N-bromo succinimide to afford crude 1-Bromomethyl-2-nitro-4-(2,2,2-trifluoro-ethoxy)-benzene which was taken to next step without any purification.

4-Methyl-3-nitrophenol was reacted with ethyl iodide to afford 4-Ethoxy-1-methyl-2-nitro-benzene in 97% yield. ¹H-NMR (400 MHz, CDCl₃) δ (ppm): 7.491 (s, 1H), 7.200-7.221 (d, 1H, J=8.4 Hz), 7.029-7.057 (dd, 1H, J=11.2 Hz, 2.4 Hz), 4.038-4.090 (q, 2H, J=6.8 Hz), 2.517 (s, 3H), 1.413-1.448 (t, 3H, J=7.2 Hz).

4-Ethoxy-1-methyl-2-nitro-benzene was reacted with N-bromo succinimide to afford crude 1-Bromomethyl-4-ethoxy-2-nitro-benzene which was taken to next step without any purification.

A solution of bromine (3.1 ml, 61.79 mmol) in 200 ml of DCM was added drop-wise to a solution of 2-trifluoromethoxyphenol (10 g, 56 mmol) in dichloromethane (100 ml) at 0° C. The mixture was stirred at room temperature for 48 h. The reaction mixture was quenched with saturated aqueous sodium thiosulfate and extracted with DCM twice. The combined organic extracts were washed with water and brine, dried over anhydrous sodium sulfate, and evaporated to afford crude 4-bromo-2-trifluoromethoxyphenol which was taken without further purification for next step. ¹H-NMR (400 MHz, DMSO-d₆) δ 10.468 (s, 1H), 7.455 (s, 1H), 7.355-7.383 (dd, 1H, J=11.2 Hz, 2.4 Hz), 6.961-6.983 (d, 1H, J=8.8 Hz) ppm. A mixture of crude 4-bromo-2-trifluoromethoxyphenol (2.0 g, 7.8 mmol), cyclopropylmethyl bromide (2.1 g, 1.6 mmol), sodium hydroxide (0.624 g, 1.6 mmol), and DMF (50 ml) was stirred for 16 h at 70° C. On cooling, water was added to the reaction mixture followed by extraction with ethyl acetate twice. The organic extracts were washed with cold water followed by brine, and were dried over anhydrous sodium sulfate. The organic layer was concentrated under reduced pressure to obtain crude product. Purification by column chromatography (silica gel, hexane:ethyl acetate, 95:5) afforded 1.8 g (74% yield) of 4-bromo-1-cyclopropylmethoxy-2-trifluoromethoxybenzene. ¹H-NMR (400 MHz, CDCl₃) δ 7.260-7.366 (m, 2H), 6.838-6.860 (d, 1H, J=8.8 Hz), 3.848-3.865 (d, 2H, J=6.8 Hz), 1.233-1.294 (m, 1H), 0.594-0.657 (m, 2H), 0.317-0.373 (m, 2H) ppm. A 1 M solution of n-butyl lithium in hexanes (8.7 ml, 8.7 mmol) was added over a period of 10 minutes at −78° C. to a pre-cooled solution of 4-bromo-1-cyclopropylmethoxy-2-trifluoromethoxybenzene (1.8 g, 5.8 mmol) in THF (20 ml). The reaction mixture was stirred at −78° C. for further 30 minutes. DMF (0.51 g, 7.0 mmol) was added to the reaction mixture over a period of 5 minutes at −78° C. The reaction mixture was stirred at the same temperature for 1.5 h. The reaction mixture was quenched with water and extracted with ethyl acetate twice. The combined organic extracts were washed with brine and dried over anhydrous sodium sulfate. The organic layer was concentrated under reduced pressure and purified by column chromatography (silica gel, hexane:ethyl acetate, 95:5) to afford 0.82 g (55% yield) of 4-cyclopropylmethoxy-3-trifluoromethoxybenzaldehyde. ¹H-NMR (400 MHz, CDCl₃) δ 9.872 (s, 1H), 7.769-7.789 (m, 2H), 7.063-7.084 (d, 1H, J=8.4 Hz), 3.977-3.994 (d, 2H, J=6.8 Hz), 0.906-0.924 (m, 1H), 0.657-0.703 (m, 2H), 0.382-0.421 (m, 2H) ppm.

Sodium borohydride (145.4 mg, 3.84 mmol) was added to an ice-cold solution of 4-cyclopropylmethoxy-3-trifluoromethoxybenzaldehyde (500 mg, 1.92 mmol) in methanol (20 ml). The reaction mixture was warmed to room temperature and stirred for 2 h. The solvent was evaporated, the reaction mass was diluted with water and extracted with ethyl acetate twice. The combined organic extracts were washed with water and brine, and dried over anhydrous sodium sulfate. The organic layer was concentrated under reduced pressure to obtain crude (4-cyclopropylmethoxy-3-trifluoromethoxyphenyl)methanol which was taken to the next step without further purification.

Carbon tetrabromide (126.5 mg, 0.381 mmol) was added to a stirred solution of crude (4-cyclopropylmethoxy-3-trifluoromethoxyphenyl)methanol (100 mg, 0.381 mmol) and triphenylphosphine (100.1 mg, 0.381 mmol) in carbon tetrachloride (10 ml) at 0° C., and the resulting mixture was stirred for 16 h at room temperature. The solvent was evaporated under vacuum and the resulting residue was purified by column chromatography (silica gel, hexane:ethyl acetate, 95:5) to afford 80 mg (64% yield) of 4-bromomethyl-1-cyclopropylmethoxy-2-trifluoromethoxybenzene as a light yellow oil.

¹H-NMR (400 MHz, CDCl₃) δ 7.237 (s, 1H), 6.907-6.928 (d, 2H, J=8.4 Hz), 4.449 (s, 2H), 3.874-3.890 (d, 2H, J=6.4 Hz), 0.865-0.941 (m, 1H), 0.610-0.658 (m, 2H), 0.341-0.380 (m, 2H) ppm.

### Final Products

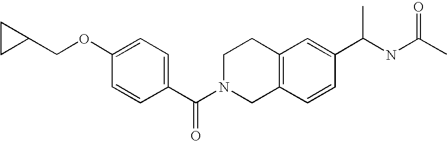

### Example 1

A mixture of 4-cyclopropylmethoxybenzoic acid (58 mg, 0.3012 mmol), EDC.HCl (86.6 mg, 0.4520 mmol), HOBt.H₂O (51 mg, 0.3313 mmol) and TEA (0.126 ml, 0.9036 mmol) was stirred in DMF (15 ml) for 15-20 min. N-[1-(1,2,3,4-Tetrahydroisoquinolin-6-yl)ethyl]acetamide trifluoroacetic acid salt (100 mg, 0.3012 mmol) in DMF (5 ml) was added to the reaction mixture at room temperature and stirred for 16 h at room temperature. Cold water was added to the reaction mixture and the reaction mass was extracted with ethyl acetate twice. The organic extracts were washed with ice-cold water followed by brine and dried over anhydrous Na₂SO₄. The solvent was evaporated under vacuum to obtain a crude residue. Purification of crude by preparative TLC (MeOH:DCM 0.5:9.5) afforded 40 mg (33.78%) of N-{1-[2-(4-cyclopropylmethoxybenzoyl)-1,2,3,4-tetrahydroisoquinolin-6-yl]ethyl}acetamide.

LCMS: [M+H]⁺393.5; 1H-NMR (DMSO): 7.875-7.895 (d, 1H, J=8 Hz)), 7.061-7.081 (d, 2H, J=8 Hz), 6.77 (s, 3H), 6.648-6.669 (d, 2H, J=8.4 Hz), 4.507-4.542 (m, 1H), 4.319 (bs, 2H), 3.541-3.558 (d, 2H, J=6.8 Hz), 3.356 (bs, 2H), 2.514 (bs, 2H), 1.500 (s, 3H), 0.977-0.993 (d, 3H, J=6.4 Hz), 0.919 (bs, 1H), 0.256-0.273 (d, 2H, J=6.8 Hz), 0.013-0.023 (d, 2H, J=4 Hz).

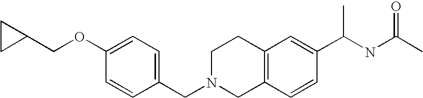

### Example 2

1-(1,2,3,4-Tetrahydroisoquinolin-6-yl)ethanone (500 mg, 2.8534 mmol), 1-bromomethyl-4-cyclopropylmethoxybenzene (826 mg, 3.4241 mmol) and K₂CO₃ (789 mg, 5.7068 mmol) were taken in DMF (10 ml) and stirred for 1.5 hrs at 70° C. The reaction mixture was diluted with water and extracted with ethyl acetate twice. The organic extracts were washed with ice-cold water, brine and dried over anhydrous Na₂SO₄. The solvent was evaporated to obtain a crude residue. Purification of crude by column chromatography (silica gel 60-120 mesh, 1.5:8.5 EtOAc:hexane) afforded 400 mg (41.78%) of 1-[2-(4-cyclopropylmethoxybenzyl)-1,2,3,4-tetrahydroisoquinolin-6-yl]ethanone. LCMS: [M+H]⁺ 336.2; 1H-NMR (CDCl₃): 7.675-7.699 (m, 2H), 7.266-7.286 (d, 2H, J=8 Hz), 7.053-7.071 (d, 1H, J=7.2 Hz), 6.869-6.887 (d, 2H, J=7.2 Hz), 3.795-3.812 (d, 2H, J=6.8 Hz), 3.646 (s, 4H), 2.940 (bs, 2H), 2.756-2.768 (m, 2H), 2.562 (s, 3H), 0.891-0.951 (m, 1H), 0.636-0.653 (d, 2H, J=6.8 Hz), 0.355 (bs, 2H). 1-[2-(4-Cyclopropylmethoxybenzyl)-1,2,3,4-tetrahydroisoquinolin-6-yl]ethanone was converted to 1-[2-(4-cyclopropylmethoxybenzyl)-1,2,3,4-tetrahydroisoquinolin-6-yl]ethylamine using the same reductive amination method employed for the preparation of Intermediate 4. LCMS: [M+H]⁺ 337.2. TEA (0.165 ml, 1.190 mmol) was added to a stirred solution of 1-[2-(4-cyclopropylmethoxybenzyl)-1,2,3,4-tetrahydroisoquinolin-6-yl]ethylamine (100 mg, 0.02972 mmol), in DCM (15 ml) at 0° C. Acetic anhydride (0.028 ml, 0.2972 mmol) was added to the reaction mixture at 0° C. and the reaction stirred for 30 min after warming to room temperature. The reaction mixture was diluted with DCM and washed with sat. NaHCO₃ solution, water and brine. The organic layer was dried over anhydrous Na₂SO₄ before evaporation of solvent to obtain a crude residue. Purification of crude by preparative HPLC afforded 30 mg (26.66%) of N-{1-[2-(4-cyclopropylmethoxybenzyl)-1,2,3,4-tetrahydroisoquinolin-6-yl]ethyl}acetamide. LCMS: [M+H]⁺ 379.5; 1H-NMR (CDCl₃): 7.261-7.281 (m, 2H), 7.041 (bs, 2H), 6.942-6.963 (d, 1H, J=8.4 Hz), 6.855-6.876 (d, 2H, J=8.4 Hz), 5.589 (bs, 1H), 5.041-5.076 (m, 1H), 3.792-3.808 (d, 2H, J=6.4 Hz), 3.573-3.604 (d, 4H, J=12.4 Hz), 2.874 (bs, 2H), 2.718-2.731 (m, 2H), 1.966 (s, 3H), 1.446-1.462 (d, 3H, J=6.4 Hz), 1.260-1.273 (m, 1H), 0.633-0.651 (d, 2H, 7.2 Hz), 0.343-0.354 (d, 2H, J=4.4 Hz).

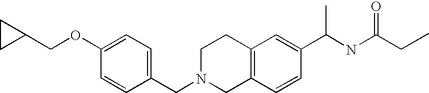

### Example 3

1-[2-(4-Cyclopropylmethoxybenzyl)-1,2,3,4-tetrahydroisoquinolin-6-yl]ethylamine was reacted with propionyl chloride using the same method employed for the acylation in the synthesis of Example 2, to afford N-{1-[2-(4-cyclopropylmethoxybenzyl)-1,2,3,4-tetrahydroisoquinolin-6-yl]ethyl}propionamide in 10% yield. LCMS: [M+H]⁺ 393.6; 1H-NMR (DMSO-d₆): 8.047-8.067 (d, 1H, J=8 Hz), 7.202-7.223 (d, 2H, J=8.4 Hz), 6.980-6.996 (m, 2H), 6.892-6.913 (d, 1H, J=8.4 Hz), 6.843-6.864 (d, 2H, J=8.4 Hz), 4.789-4.825 (m, 1H), 3.767-3.783 (d, 2H, J=6.4 Hz), 3.529 (s, 2H), 3.439 (s, 2H), 2.738-2.753 (m, 2H), 2.602-2.631 (t, 2H, J=6 Hz), 2.041-2.097 (q, 2H, J=7.6 Hz), 1.263-1.280 (d, 3H, J=6.8 Hz), 1.172-1.221 (m, 1H), 0.937-0.975 (t, 3H, J=7.6 Hz), 0.522-0.566 (m, 2H), 0.275-0.312 (m, 2H).

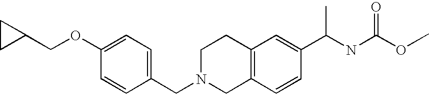

### Example 4

[1-(1,2,3,4-Tetrahydroisoquinolin-6-yl)ethyl]carbamic acid methyl ester trifluoroacetic acid salt was reacted with bromomethyl-4-cyclopropylmethoxybenzene via the same method used for Example 2 in order to afford {1-[2-(4-cyclopropylmethoxybenzyl)-1,2,3,4-tetrahydroisoquinolin-6-yl]ethyl}carbamic acid methyl ester in 10% yield. LCMS: [M+H]⁺ 395.2; 1H-NMR (DMSO): 7.544 (bs, 1H), 7.204-7.224 (d, 2H, J=8 Hz), 6.988 (s, 2H), 6.892-6.912 (d, 1H, J=8 Hz), 6.844-6.865 (d, 2H, J=8.4 Hz), 4.528-4.563 (m, 1H), 3.768-3.784 (d, 2H, J=6.4 Hz), 3.531 (s, 2H), 3.471 (s, 3H), 3.440 (s, 2H), 2.753 (bs, 2H), 2.619 (bs, 2H), 1.263-1.280 (d, 3H, J=6.8 Hz), 0.838 (bs, 1H), 0.536-0.552 (m, 2H), 0.289-0.299 (m, 2H).

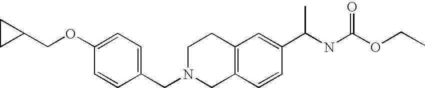

### Example 5

6-(1-Aminoethyl)-3,4-dihydro-1H-isoquinoline-2-carboxylic acid tert-butyl ester was reacted with ethyl chloroformate in the same way as employed for the N-acylation in the synthesis of Intermediate 4 to afford 6-(1-ethoxycarbonylaminoethyl)-3,4-dihydro-1H-isoquinoline-2-carboxylic acid tert-butyl ester in 75% yield. LCMS: [M-56]⁻293.4. 6-(1-Ethoxycarbonylaminoethyl)-3,4-dihydro-1H-isoquinoline-2-carboxylic acid tert-butyl ester was reacted with TFA in the same way as employed for the N-deprotection in the synthesis of Intermediate 4 to afford [1-(1,2,3,4-tetrahydroisoquinolin-6-yl)ethyl]carbamic acid ethyl ester trifluoroacetic acid salt in 40% yield. LCMS: [M+H]⁺ 249.4. [1-(1,2,3,4-Tetrahydroisoquinolin-6-yl)ethyl]carbamic acid ethyl ester trifluoroacetic acid salt was reacted with bromomethyl-4-cyclopropylmethoxybenzene via the same method used for Example 2 in order to afford {1-[2-(4-cyclopropylmethoxybenzyl)-1,2,3,4-tetrahydroisoquinolin-6-yl]ethyl}carbamic acid ethyl ester in 20% yield. LCMS: [M+H]⁺ 409.6; 1H-NMR (CDCl₃): 7.259-7.291 (m, 2H), 7.030 (s, 2H), 6.935-6.955 (d, 1H, J=8 Hz), 6.856-6.877 (d, 2H, J=8.4 Hz), 4.757-4.820 (m, 2H), 4.083-4.100 (m, 2H), 3.792-3.810 (d, 2H, J=7.2 Hz), 3.592-3.622 (d, 4H, J=12 Hz), 2.881 (bs, 2H), 2.737 (bs, 2H), 1.430-1.446 (d, 3H, J=6.4 Hz), 1.199-1.234 (t, 3H, J=6.8 Hz), 0.882-0.892 (m, 1H), 0.630-0.649 (m, 2H), 0.342-0.353 (m, 2H).

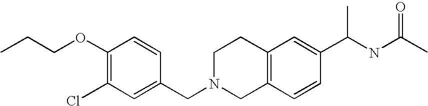

### Example 6

N-[1-(1,2,3,4-Tetrahydroisoquinolin-6-yl)ethyl]acetamide trifluoroacetic acid salt (100 mg, 0.3012 mmol), 4-bromomethyl-2-chloro-1-propoxybenzene (87.4 mg, 0.3313 mmol), CuI (57.37 mg, 0.3012 mmol) and Cs₂CO₃ (295 mg, 0.9036 mmol) were taken in DMF (8 ml) and stirred for 1 h at 70° C. under microwave conditions. The reaction mixture was cooled and poured into water and extracted with ethyl acetate twice. The combined organic extracts were washed with water, brine and dried over anhydrous Na₂SO₄. The solvent was evaporated to obtain a crude residue. Purification of the crude through column chromatography (silica gel 60-120 mesh, EtOAc) afforded 30 mg (24.84%) of N-{1-[2-(3-chloro-4-propoxybenzyl)-1,2,3,4-tetrahydroisoquinolin-6-yl]ethyl}acetamide. LCMS: [M+H]⁺ 401.6; ¹H-NMR (CDCl₃), 6 (ppm): 7.389-7.394 (d, 1H, J=2 Hz), 7.187-7.208 (d, 1H, J=8.4 Hz), 7.050-7.066 (m, 2H), 6.948-6.970 (d, 1H, J=8.8 Hz), 6.860-6.881 (d, 1H, J=8.4 Hz), 5.576-5.593 (d, 1H, J=6.8 Hz), 5.044-5.081 (m, 1H), 3.973-4.006 (t, 2H, J=6.4 Hz), 3.587 (s, 4H), 2.874-2.902 (t, 2H, J=5.6 Hz), 2.717-2.747 (t, 2H, J=6.4 Hz), 1.968 (s, 3H), 1.833-1.886 (m, 2H), 1.449-1.467 (d, 3H, J=7.2 Hz), 1.052-1.089 (t, 3H, J=7.6 Hz).

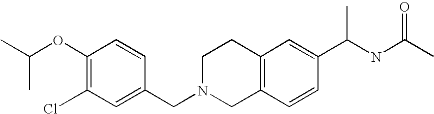

### Example 7

### N-[1-(1,2,3,4-Tetrahydroisoquinolin-6-yl)ethyl]acetamide trifluoroacetic acid salt was reacted with 4-bromomethyl-2-chloro-1-isopropoxybenzene via the same method used for Example 6 in order to obtain N-{1-[2-(3-chloro-4-isobutoxybenzyl)-1,2,3,4-tetrahydroisoquinolin-6-yl]ethyl}acetamide in 30% yield. LCMS: [M+H]⁺ 401.6; 1H-NMR (CDCl₃) 6 (ppm): 7.386-7.391 (d, 1H, J=2 Hz), 7.173-7.194 (d, 1H, J=8.4 Hz), 7.051-7.066 (m, 2H), 6.954-6.974 (d, 1H, J=8 Hz), 6.888-6.909 (d, 1H, J=8.4 Hz), 5.565-5.583 (d, 1H, J=7.2 Hz), 5.047-5.082 (m, 1H), 4.501-4.562 (m, 1H), 3.581 (s, 4H), 2.875-2.902 (t, 2H, J=5.6 Hz), 2.717-2.746 (t, 2H, J=5.6 Hz), 1.969 (s, 3H), 1.450-1.468 (d, 3H, J=7.2 Hz), 1.371-1.386 (d, 6H, J=6 Hz).

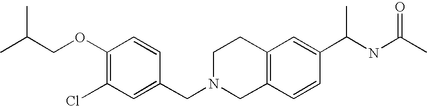

### Example 8

N-[1-(1,2,3,4-Tetrahydroisoquinolin-6-yl)ethyl]acetamide trifluoroacetic acid salt was reacted with 4-bromomethyl-2-chloro-1-isobutoxybenzene via the same method used for Example 6 in 20% yield. LCMS: [M+H]⁺ 415.7; ¹HNMR (CDCl₃), δ (ppm):: 7.387-7.392 (d, 1H, J=2 Hz), 7.180-7.200 (d, 1H, J=8 Hz), 7.048-7.064 (m, 2H), 6.948-6.969 (d, 1H, J=8.4 Hz), 6.844-6.865 (d, 1H, J=8.4 Hz), 5.569-5.584 (d, 1H, J=6 Hz), 5.046-5.098 (m, 1H), 3.772-3.789 (d, 2H, J=6.8 Hz), 3.579 (s, 4H), 2.8702-2.898 (t, 2H, J=5.6 Hz), 2.709-2.738 (t, 2H, J=6 Hz), 2.116-2.183 (m, 1H), 1.968 (s, 3H), 1.449-1.467 (d, 3H, J=7.2 Hz), 1.052-1.068 (d, 6H, J=6.4 Hz).

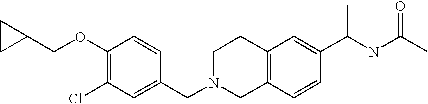

### Example 9

### N-[1-(1,2,3,4-Tetrahydroisoquinolin-6-yl)ethyl]acetamide TFA salt was reacted with 4-bromomethyl-2-chloro-1-cyclopropylmethoxybenzene via the same method used for Example 6 to afford 40% of N-{1-[2-(3-chloro-4-cyclopropylmethoxybenzyl)-1,2,3,4-tetrahydroisoquinolin-6-yl]ethyl}acetamide. LC/MS [M+H]⁺ 413.2; ¹H-NMR (400 MHz, DMSO-D6) δ (ppm): 8.156-8.138 (d, 1H, J=7.2 Hz), 7.354 (s, 1H), 7.226-7.206 (d, 1H, J=8 Hz), 7.064-7.043 (d, 1H, J=8.4 Hz), 6.989 (br, 2H), 6.927-6.907 (d, 1H, J=8 Hz), 4.819-4.784 (m, 1H), 3.892-3.876 (d, 2H, J=6.4 Hz), 3.543 (s, 2H), 3.453 (s, 2H), 2.766 (br, 2H), 2.633 (br, 2H), 1.794 (s, 3H), 1.280-1.263 (d, 3H, J=6.8 Hz), 0.839-0.822 (m, 1H), 0.571-0.553 (d, 2H, J=7.2 Hz), 0.334-0.325 (d, 2H, J=3.6 Hz).

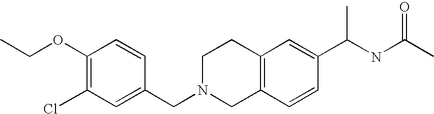

### Example 10

N-[1-(1,2,3,4-Tetrahydroisoquinolin-6-yl)ethyl]acetamide TFA salt was reacted with 4-bromomethyl-2-chloro-1-ethoxybenzene via the same method used for Example 6 to afford 33% of N-{1-[2-(3-chloro-4-ethoxybenzyl)-1,2,3,4-tetrahydroisoquinolin-6-yl]ethyl}acetamide. LC/MS [M+H]⁺ 387.1; ¹H-NMR (400 MHz, CDCl₃) δ (ppm): 7.399-7.395 (d, 1H, J=1.6 Hz), 7.214-7.194 (d, 1H, J=8 Hz), 7.067-7.052 (m, 2H), 6.970-6.949 (d, 1H, J=8.4 Hz), 6.885-6.864 (d, 1H, J=8.4 Hz), 5.587-5.570 (d, 1H, J=6.8 Hz), 5.0893-5.047 (m, 1H), 4.134-4.082 (q, 2H, J=7.2 Hz), 3.588 (s, 4H), 2.890-2.883 (t, 2H, J=6.8 Hz), 2.747-2.719 (t, 2H, J=5.6 Hz), 1.968 (s, 3H), 1.466-1.415 (m, 6H).

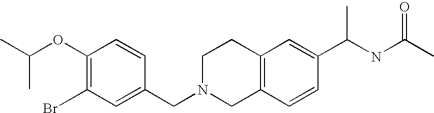

### Example 11

N-[1-(1,2,3,4-Tetrahydroisoquinolin-6-yl)ethyl]acetamide TFA salt was reacted with 2-bromo-4-bromomethyl-1-isopropoxybenzene via the same method used for Example 6 to afford 40% of N-{1-[2-(3-bromo-4-isopropoxybenzyl)-1,2,3,4-tetrahydroisoquinolin-6-yl]ethyl}acetamide. LC/MS [M+H]⁺ 445.5; ¹H-NMR (400 MHz, CDCl₃) δ (ppm): 7.564 (s, 1H), 7.224 (br, 1H), 7.052 (br, 2H), 6.975-6.956 (d, 1H, J=7.6 Hz), 6.879-6.858 (d, 1H, J=8.4 Hz), 5.588 (br, 1H), 5.082-5.048 (m, 1H), 4.550-4.521 (m, 1H), 3.580 (s, 4H), 2.888 (br, 2H), 2.729 (br, 2H), 1.968 (s, 3H), 1.467-1.450 (d, 3H, J=6.8 Hz), 1.389-1.374 (d, 6H, J=6 Hz).

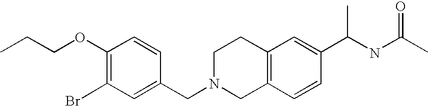

### Example 12

### N-[1-(1,2,3,4-Tetrahydroisoquinolin-6-yl)ethyl]acetamide TFA salt was reacted with 2-bromo-4-bromomethyl-1-propoxybenzene via the same method used for Example 6 to afford N-{1-[2-(3-Bromo-4-propoxybenzyl)-1,2,3,4-tetrahydroisoquinolin-6-yl]ethyl}acetamide in 18% yield. LC/MS [M+H]⁺ 445.6; ¹H-NMR (400 MHz, DMSO₆) δ (ppm): 8.14-8.16 (d, 1H, J=8 Hz), 7.51 (s, 1H), 7.25-7.28 (dd, 1H, J=8.4 Hz), 7.02-7.04 (d, 2H, J=8.4 Hz), 6.98 (s, 1H), 6.91-6.93 (d, 1H, J=8 Hz), 4.78-4.82 (m, 1H). 3.96-3.99 (t, 2H, J=6.4 Hz), 3.54 (s, 2H), 3.45 (s, 2H), 2.75-2.76 (m, 2H), 2.63-2.71 (m, 2H), 1.79 (s, 3H), 1.68-1.75 (m, 2H), 1.26-1.28 (d, 3H, J=6.4 Hz), 0.97-1.01 (tr, 3H, J=6.8 Hz).

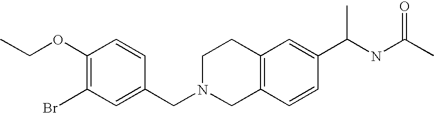

### Example 13

N-[1-(1,2,3,4-Tetrahydroisoquinolin-6-yl)ethyl]acetamide TFA salt was reacted with 2-bromo-4-bromomethyl-1-propoxybenzene via the same method used for Example 6 to afford N-{1-[2-(3-bromo-4-propoxybenzyl)-1,2,3,4-tetrahydroisoquinolin-6-yl]ethyl}acetamide in 18% yield. LC/MS [M+H]⁺ 431.6; ¹H-NMR (400 MHz, DMSO₆) δ (ppm): 8.14-8.16 (d, 1H, J=8 Hz), 7.51 (s, 1H), 7.25-7.28 (dd, 1H, J=8.4 Hz), 7.02-7.04 (d, 2H, J=8.4 Hz), 6.98 (s, 1H), 6.91-6.93 (d, 1H, J=8 Hz), 4.78-4.82 (m, 1H). 4.05-4.10 (m, 2H), 3.54 (s, 2H), 3.45 (s, 2H), 2.71-2.78 (m, 2H), 2.62-2.64 (m, 2H), 1.79 (s, 3H), 1.31-1.35 (tr, 3H, J=6.8 Hz).1.26-1.28 (d, 3H, J=6.8 Hz).

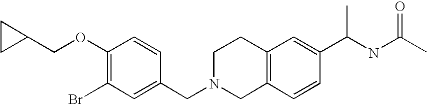

### Example 14

N-[1-(1,2,3,4-Tetrahydroisoquinolin-6-yl)ethyl]acetamide TFA salt was reacted with 2-bromo-4-bromomethyl-1-cyclopropylmethoxybenzene via the same method used for Example 6 to afford N-{1-[2-(3-bromo-4-cyclopropylmethoxybenzyl)-1,2,3,4-tetrahydroisoquinolin-6-yl]ethyl}acetamide in 13% yield. LC/MS [M+H]⁺ 457.1; ¹H-NMR (400 MHz, DMSO₆) δ (ppm): 8.14-8.16 (d, 1H, J=8 Hz), 7.51 (s, 1H), 7.24-7.26 (d, 1H, J=8.4 Hz), 7.01-7.03 (d, 2H, J=8.4 Hz), 6.98 (s, 1H), 6.90-6.92 (d, 1H, J=8 Hz), 4.78-4.82 (m, 1H), 3.87-3.89 (d, 2H, J=6.8 Hz), 3.54 (s, 2H), 3.45 (s, 2H), 2.76 (s, 2H), 2.63-2.64 (m, 2H), 1.79 (s, 3H), 1.28-1.32 (d, 3H, J=16.4 Hz), 1.19-1.22 (m, 1H), 0.55-0.56 (m, 2H), 0.33-0.34 (m, 2H).

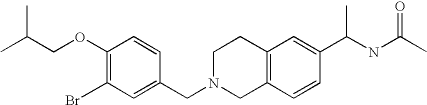

### Example 15

### N-[1-(1,2,3,4-Tetrahydroisoquinolin-6-yl)ethyl]acetamide TFA salt was reacted with 2-bromo-4-bromomethyl-1-isobutoxybenzene via the same method used for the alkylation step in the synthesis of Intermediate 5 to afford N-{1-[2-(3-bromo-4-isobutoxybenzyl)-1,2,3,4-tetrahydroisoquinolin-6-yl]ethyl}acetamide in 15% yield. LC/MS [M+H]⁺ 459.1; ¹H-NMR (400 MHz, DMSO₆) δ (ppm): 8.14-8.16 (d, 1H, J=8 Hz), 7.51 (s, 1H), 7.25-7.27 (d, 1H, J=8.4 Hz), 7.01-7.04 (d, 2H, J=8.4 Hz), 6.99 (s, 1H), 6.91-6.93 (d, 1H, J=8 Hz), 4.78-4.82 (m, 1H), 3.79-3.80 (d, 2H, J=6 Hz), 3.54 (s, 2H), 3.45 (s, 2H), 2.76 (s, 2H), 2.63-2.64 (m, 2H), 1.99-2.04 (m, 1H) 1.79 (s, 3H), 1.26-1.28 (d, 3H, J=6.8 Hz), 0.98-1.00 (d, 6H, J=6.4 Hz).

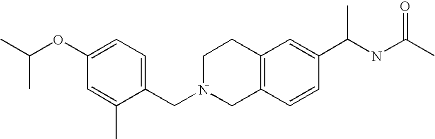

### Example 16

N-[1-(1,2,3,4-Tetrahydroisoquinolin-6-yl)ethyl]acetamide TFA salt was reacted with 1-bromomethyl-4-isopropoxy-2-methylbenzene via the same method used for Example 6 to afford N-{1-[2-(4-isopropoxy-2-methylbenzyl)-1,2,3,4-tetrahydroisoquinolin-6-yl]ethyl}acetamide in 15% yield. LC/MS [M+H]⁺ 381.5; ¹HNMR (400 MHz, CDCl₃), δ (ppm): 7.173-7.194 (d, 1H, J=8.4 Hz), 7.039 (s, 2H), 6.952-6.972 (d, 1H, J=8 Hz), 6.664-6.709 (m, 2H), 5.639-5.656 (d, 1H, J=6.8 Hz), 5.040-5.075 (t, 1H, J=6.8 Hz), 4.496-4.555 (m, 1H), 3.563-3.586 (d, 4H, J=9.2 Hz), 2.839-2.853 (d, 2H, J=5.6 Hz), 2.632-2.731 (m, 2H), 2.355 (s, 3H), 2.040(s, 1H), 1.895-2.004(m, 3H), 1.446-1.462 (d, 3H, J=6.4 Hz), 1.320-1.335 (d, 6H, J=6 Hz).

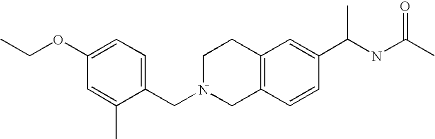

### Example 17

N-[1-(1,2,3,4-Tetrahydroisoquinolin-6-yl)ethyl]acetamide TFA salt was reacted with 1-bromomethyl-4-ethoxy-2-methylbenzene via the same method used for Example 6 to afford 40% of N-{1-[2-(4-ethoxy-2-methylbenzyl)-1,2,3,4-tetrahydroisoquinolin-6-yl]ethyl}acetamide. LC/MS [M+H]⁺ 367.5; ¹H-NMR (400 MHz, DMSO-D6) δ (ppm): 9.707 (broad s, 1H, 8.246-8.227 (d, 1H, J=7.6 Hz), 7.405-7.385 (d, 1H, J=8 Hz), 7.15-7.134 (m, 3H), 6.861-6.839 (m, 2H), 4.846-4.811 (m, 1H), 4.382-4.324 (broad d, 4H), 4.061-4.027 (q, 2H), 3.416 (br, 2H), 3.060 (br, 2H), 2.364 (s, 3H), 1.803 (s, 3H) 1.331-1.275 (m, 6H).

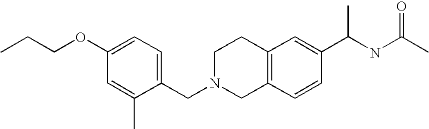

### Example 18

### N-[1-(1,2,3,4-Tetrahydroisoquinolin-6-yl)ethyl]acetamide triflate salt was reacted with 1-bromomethyl-2-methyl-4-propoxybenzene via the same method used for Example 6 to afford 5% of N-{1-[2-(2-methyl-4-propoxybenzyl)-1,2,3,4-tetrahydroisoquinolin-6-yl]ethyl}acetamide. LC/MS [M+H]⁺ 381.5; ¹HNMR (400 MHz, CDCl₃) δ (ppm): 7.177-7.198 (d, 1H, J=8.4 Hz), 7.037 (s, 2H), 6.949-6.968 (d, 1H, J=7.6 Hz), 6.675-6.742 (m, 2H), 3.890-3.922 (t, 2H, J=6 Hz), 3.557-3.575 (d, 4H, J=7.2 Hz), 2.833-2.845 (d, 2H, J=4.8 Hz), 2.640-2.716 (m, 2H), 2.344 (s, 3H), 2.002-2.1199 (m, 1H), 1.961 (s, 3H), 1.787-1.895 (m, 2H), 1.446-1.463 (d, 3H, J=6.8 Hz), 1.257 (s, 1H), 1.013-1.049 (t, 3H, J=7.2 Hz).

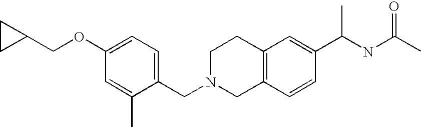

### Example 19

N-[1-(1,2,3,4-Tetrahydroisoquinolin-6-yl)ethyl]acetamide triflate salt and 1-bromomethyl-4-cyclopropylmethoxy-2-methylbenzene were reacted via the same method used for Example 6 to obtain 4-[2-(4-cyclopropylmethoxy-2-methylbenzyl)-1,2,3,4-tetrahydroisoquinolin-6-yl]-pentan-2-one in 10% yield. LC/MS [M+H]⁺ 393.7; ¹HNMR (400 MHz, CDCl₃) δ (ppm): 7.180-7.200 (d, 1H, J=8 Hz), 7.040-7.058 (d, 2H, J=7.2 Hz), 6.950-6.970 (d, 1H, J=8 Hz), 6.733 (s, 1H), 6.672-6.699 (d, 1H, J=8.4 Hz), 5.565-5.583 (d, 1H, J=7.2 Hz), 5.043-5.079 (m, 1H), 3.780-3.796 (d, 2H, J=6.4 Hz), 3.557-3.576 (d, 4H, J=7.6 Hz), 2.834-2.862 (t, 2H, J=5.6 Hz), 2.688-2.716 (t, 2H J=5.6 Hz), 2.343 (s, 3H), 1.964(s, 3H), 1.449-1.469 (d, 3H, J=7.2 Hz), 1.236-1.297 (m, 2H), 0.885-0.947 (m, 1H), 0.610-0.657 (m, 2H), 0.322-0.360 (m, 2H).

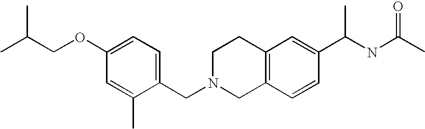

### Example 20

N-[1-(1,2,3,4-Tetrahydroisoquinolin-6-yl)ethyl]acetamide triflate salt was reacted with 1-bromomethyl-4-isobutoxy-2-methylbenzene via the same method used for Example 6 to afford 35% of N-{1-[2-(4-isopropoxy-2-methylbenzyl)-1,2,3,4-tetrahydroisoquinolin-6-yl]ethyl}acetamide. LC/MS [M+H]⁺ 395.7; ¹HNMR (400 MHz, CDCl₃) δ (ppm): 7.176-7.197 (d, 1H, J=8.4 Hz), 7.040-7.059 (d, 2H, J=7.6 Hz), 6.972-6.993 (d, 2H, J=8.4 Hz), 5.565-5.582 (d, 1H, J=6.8 Hz), 5.043-5.079 (m, 1H), 3.696-3712 (d, 2H, J=6.4 Hz), 3.556-3.577 (d, 4H, J=8.4 Hz), 2.8342-847 (d, 2H, J=5.2 Hz), 2.687-2.715 (m, 2H), 2.345 (s, 3H), 2.036-2.086 (m, 1H), 1.963 (s, 3H), 1.449-1.466 (d, 3H, J=6.8 Hz), 1.259 (bs, 1H), 0.978-1.010 (d, 6H, J=12.8 Hz).

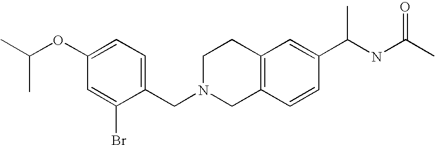

### Example 21

N-[1-(1,2,3,4-Tetrahydroisoquinolin-6-yl)ethyl]acetamide TFA salt was reacted with 2-bromo-1-bromomethyl-4-isopropoxybenzene (102 mg, 0.33 mmol), via the same method used for Example 6 to afford 46% of N-{1-[2-(2-bromo-4-isopropoxybenzyl)-1,2,3,4-tetrahydroisoquinolin-6-yl]ethyl}acetamide. LC/MS [M+H]⁺ 445.3; ¹H-NMR (400 MHz, DMSO-d₆) δ (ppm): 8.247-8.228 (d, 1H, J=7.6 Hz), 7.618-7.596 (d, 1H, J=8.8 Hz), 7.285 (s, 1H), 7.132-7.072 (m, 4H), 4.848-4.813 (m, 1H), 4.721-4.691 (m, 1H), 4.497 (s, 2H), 4.386 (s, 2H), 3.472 (br, 2H), 3.070 (broad s, 2H), 1.803 (s, 3H), 1.293-1.222 (m, 9H).

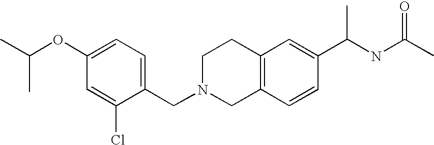

### Example 22

N-[1-(1,2,3,4-Tetrahydroisoquinolin-6-yl)ethyl]acetamide TFA salt and 1-bromomethyl-2-chloro-4-isopropoxybenzene were reacted via the same method used for Example 6 to afford 45% of N-{1-[2-(2-chloro-4-isopropoxybenzyl)-1,2,3,4-tetrahydroisoquinolin-6-yl]ethyl}acetamide. LC/MS [M+H]⁺ 401.2; ¹H-NMR (400 MHz, DMSO-d₆) δ (ppm): 8.243-8.224 (d, 1H, J=7.6), 7.608-7.587 (d, 1H, J=8.4), 7.152-7.134 (broad, 4H), 7.047-7.026 (d, 1H J=8.4), 4.847-4.812 (m, 1H), 4.722-4.694 (m, 1H), 4.489 (s, 2H), 4.371 (s, 2H), 3.452 (broad s, 2H), 3.065 (broad s, 2H), 1.802 (s, 3H), 1.290-1.262 (m, 9H).

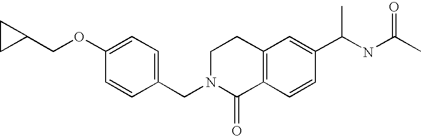

### Example 23

To a suspension of sodium hydride (0.12 g, 5.0 mmol) in dry THF at 0° C. was added 6-methoxy-3,4-dihydro-2H-isoquinolin-1-one (0.3 g, 1.69 mmol) and the reaction mixture was refluxed for 1 h. 1-Bromomethyl-4-cyclopropylmethoxybenzene (0.43 g, 2.0 mmol) was then added and the reaction mixture was heated at 65° C. overnight. The reaction mass was cooled to room temperature, water was added and the layers were separated. The aqueous layer was extracted with ethyl acetate. The ethyl acetate layer was washed with brine before drying over anhydrous sodium sulphate. The solvent was evaporated to obtain a residue. Purification of the residue using column chromatography (ethyl acetate:hexane 1:1) afforded 0.22 g (42.3% yield) of 2-(4-cyclopropylmethoxybenzyl)-6-methoxy-3,4-dihydro-2H-isoquinolin-1-one. LC/MS [M+H]⁺ 338.5; ¹H-NMR (400 MHz, CDCl₃) δ (ppm): 8.01-8.10 (d, 1H, J=8.4 Hz), 7.91-7.93 (d, 2H, J=8.0 Hz), 7.41-7.43 (d, 2H, J=8.0 Hz), 6.86-6.88 (d, 1H, J=8.0 Hz), 6.66 (s, 1H), 4.82 (s, 2H), 3.84 (s, 3H), 3.46-3.49 (t, 2H), 2.91-2.94 (t, 2H), 2.58 (s, 3H), 1.84 (s, 3H), 1.39-1.43 (d, 3H, J=16 Hz), 1.27-1.28 (d, 6H, J=5.6 Hz). A solution of 2-(4-cyclopropylmethoxybenzyl)-6-methoxy-3,4-dihydro-2H-isoquinolin-1-one (0.5 g, 1.5 mmol) and sodium methanethiolate (0.21 g, 3.0 mmol) in DMF was heated at 150° C. for 1.5 h. The reaction mass was cooled to room temperature, water was added and the organic layer was separated. The aqueous layer was extracted with ethyl acetate. The ethyl acetate layer was washed with brine before drying over anhydrous sodium sulphate. The solvent was evaporated to afforded 0.41 g (85.4% yield) of 2-(4-cyclopropylmethoxybenzyl)-6-hydroxy-3,4-dihydro-2H-isoquinolin-1-one. LC/MS [M+H]⁺ 324.4. The solution of 2-(4-cyclopropylmethoxybenzyl)-6-hydroxy-3,4-dihydro-2H-isoquinolin-1-one (0.4 g, 1.23 mmol) and Et₃N (0.187 g, 1.85 mmol) in DCM was cooled to 0° C. Triflic anhydride (0.32 g, 1.35 mmol) was added and the reaction mixture was stirred at room temperature for 1 h. The reaction mass was diluted with ice-cooled water and the layers were separated. The aqueous layer was extracted with DCM. The DCM layer was washed with brine before drying over anhydrous sodium sulphate. The solvent was evaporated to afforded 0.42 g (75% yield) of trifluoromethanesulfonic acid 2-(4-cyclopropylmethoxybenzyl)-1-oxo-1,2,3,4-tetrahydroisoquinolin-6-yl ester. LC/MS [M+H]⁺ 456.1. A solution of trifluoromethanesulfonic acid 2-(4-cyclopropylmethoxybenzyl)-1-oxo-1,2,3,4-tetrahydroisoquinolin-6-yl ester (0.42 g, 0.992 mmol), TEA (0.11 g, 1.1 mmol) in DMF was flushed with argon for 10 min. Palladium acetate (0.020 g 0.0922 mmol), 1,3-bis(diphenylphosphino)propane (0.045 g, 0.110 mmol) and vinyl-1-butylether was added sequentially after which the reaction mixture was heated at 90° C. overnight in a sealed tube. The reaction mass was cooled to room temperature, the insolubles filtered through Celite and the filtrate was diluted with water and extracted with ethyl acetate. The ethyl acetate layer was washed with brine before drying over anhydrous sodium sulphate. The solvent was evaporated to obtain a residue. Purification of the residue by column chromatography (silica gel 60-120 mesh, EtOAc:hexane 3.5:6.5) afforded a sticky solid. This sticky solid was further stirred with conc. HCl (5 ml) for 1 h. The solution was neutralized with sodium bicarbonate solution and extracted with ethyl acetate. The ethyl acetate layer was washed with brine before drying over anhydrous sodium sulphate to afford 0.22 g (68.3% yield) of 6-acetyl-2-(4-cyclopropylmethoxybenzyl)-3,4-dihydro-2H-isoquinolin-1-one. LC/MS [M+H]⁺ 350.4. 6-Acetyl-2-(4-cyclopropylmethoxybenzyl)-3,4-dihydro-2H-isoquinolin-1-one was converted to 6-(1-aminoethyl)-2-(4-cyclopropylmethoxybenzyl)-3,4-dihydro-2H-isoquinolin-1-one using the same method as for the reductive amination in the synthesis of Intermediate 4, and used in next step without any purification. LC/MS [M+H]⁺ 351.5. 6-(1-Amino ethyl)-2-(4-cyclopropylmethoxybenzyl)-3,4-dihydro-2H-iso quinolin-1-one was converted in 18% yield to N-{1-[2-(4-cyclopropylmethoxybenzyl)-1-oxo-1,2,3,4-tetrahydroisoquinolin-6-yl]ethyl}acetamide via the same method used for the acylation in the synthesis of Example 6. LC/MS [M+H]⁺ 393.2; ¹H-NMR (400 MHz, CDCl₃) δ (ppm): 8.08-8.10 (d, 1H, J=8.0 Hz), 7.21-7.280 (m, 3H), 7.09 (s, 1H), 6.83-6.85 (d, 2H, J=8.0 Hz), 5.72-5.73 (broad, 1H), 5.10-5.13 (t, 1H), 4.7 (s, 2H), 3.77-3.79 (d, 2H, J=8), 3.42-3.45 (t, 2H), 2.89 (s, 3H), 1.99 (s, 3H) 1.46-1.48 (d, 3H, J=8), 0.62-0.64 (d, 2H, J=8), 0.33-0.34 (d, 2H, J=4 Hz).

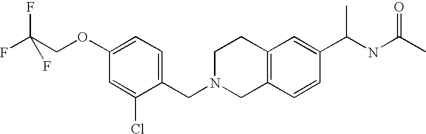

### Example 24

N-[1-(1,2,3,4-Tetrahydro-isoquinolin-6-yl)-ethyl]-acetamide TFA salt and 1-Bromomethyl-2-chloro-4-(2,2,2-trifluoro-ethoxy)-benzene were reacted to afford N-(1-{2-[2-Chloro-4-(2,2,2-trifluoro-ethoxy)-benzyl]-1,2,3,4-tetrahydro-isoquinolin-6-yl}-ethyl)-acetamide in 40% yield. LC/MS [M+H]+=441.6, ¹H-NMR (400 MHz, DMSO-D₆) δ (ppm): 8.142-8.162 (d, 1H, J=8 Hz), 7.441-7.462 (d, 1H, J=8.4 Hz), 7.186 (s, 1H), 6.996-7.043 (m, 3H), 6.923-6.944 (d, 1H, J=8.4), 4.755-4.823 (m, 3H), 3.655 (s, 2H), 3.536 (s, 2H), 2.760 (bs, 2H), 2.663-2.691 (t, 2H, J=6.4 Hz), 1.796 (s, 3H), 1.266-1.284 (d, 3H, J=7.2 Hz).

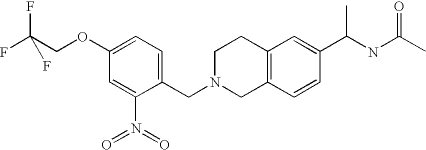

### Example 25

N-[1-(1,2,3,4-Tetrahydro-isoquinolin-6-yl)-ethyl]-acetamide TFA salt and 1-Bromomethyl-2-nitro-4-(2,2,2-trifluoro-ethoxy)-benzene were reacted to afford N-(1-{2-[2-Nitro-4-(2,2,2-trifluoro-ethoxy)-benzyl]-1,2,3,4-tetrahydro-isoquinolin-6-yl}-ethyl)-acetamide in 36% yield. LC/MS [M+H]+=452.7, ¹H-NMR (400 MHz, DMSO-D₆) δ (ppm): 8.131-8.152 (d, 1H, J=8.4 Hz), 7.587-7.615 (m, 2H), 7.344-7.372 (dd, 1H, J=11.2 Hz, 2.8 Hz), 6.979-7.008 (m, 2H), 6.904-6.923 (d, 1H, J=7.8 Hz), 4.779-4.913 (m, 3H), 3.804 (s, 2H), 3.474 (s, 3H), 2.671-2.697 (t, 2H, J=5.2 Hz), 2.551-2.579 (t, 2H, J=5.6 Hz), 1.792 (s, 3H), 1.263-1.280 (d, 3H, J=6.8 Hz).

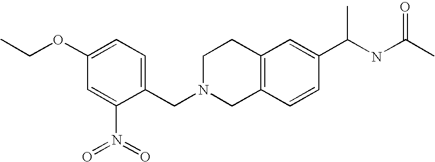

### Example 26

### N-[1-(1,2,3,4-Tetrahydro-isoquinolin-6-yl)-ethyl]-acetamide TFA salt and 1-Bromomethyl-4-ethoxy-2-nitro-benzene were reacted to afford N-{1-[2-(4-Ethoxy-2-nitro-benzyl)-1,2,3,4-tetrahydro-isoquinolin-6-yl]-ethyl}-acetamide as off-white semi-solid. LC/MS [M+H]+=398.8, ¹H-NMR (400 MHz, DMSO-D₆) δ (ppm): 8.130-8.151 (d, 1H, J=8.4 Hz), 7.515-7.536 (d, 1H, J=8.4 Hz), 7.375 (s, 1H), 7.193-7.221 (dd, 1H, J=11.2 Hz, 2.4 Hz), 6.977-7.006 (m, 2H), 6.903-6.923 (d, 1H, J=8 Hz), 4.777-4.814 (m, 1H), 4.069-4.122 (q, 2H, J=7.2 Hz), 3.777 (s, 2H), 3.464 (s, 2H), 2.669-2.682 (bs, 2H), 2.544-2.573 (t, 2H, J=6 Hz), 1.791 (s, 3H), 1.307-1.342 (t, 3H, J=6.8 Hz), 1.262-1.279 (d, 3H, J=6.8 Hz).

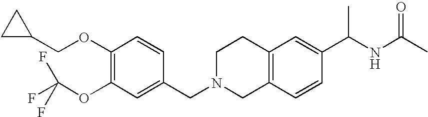

### Example 27

A mixture of N-[1-(1,2,3,4-tetrahydroisoquinolin-6-yl)ethyl]acetamide trifluoroacetic acid salt (82 mg, 0.246 mmol), 4-bromomethyl-1-cyclopropylmethoxy-2-trifluoromethoxybenzene (80 mg, 0.246 mmol), cesium carbonate (160 mg, 0.492 mmol), cuprous iodide (47 mg, 0.246 mmol), and DMF (6 ml) was stirred for 2 h at 70° C. under microwave conditions. On cooling, the reaction mixture was diluted with water and extracted with ethyl acetate twice. The combined organic extracts were washed with ice-cold water and brine, followed by drying over anhydrous sodium sulfate. The organic layer was concentrated under reduced pressure and purified by preparative thin layer chromatography (ethyl acetate:hexane, 70:30) to afford 40 mg of N-{1-[2-(4-cyclopropylmethoxy-3-trifluoromethoxybenzyl)-1,2,3,4-tetrahydroisoquinolin-6-yl]ethyl}acetamide in 35% yield. ¹H-NMR (400 MHz, DMSO-d₆) δ 9.996 (bs, 1H), 8.223-8.243 (d, 1H, J=8 Hz), 7.576 (s, 1H), 7.484-7.505 (d, 1H, J=8.4 Hz), 7.302-7.323 (d, 1H, J=8.4 Hz), 7.136-7.170 (m, 2H), 4.809-4.846 (m, 1H), 4.424 (bs, 2H), 4.276 (bs, 2H), 3.962-3.980 (d, 2H, J=7.2 Hz), 3.417 (bs, 2H), 3.057 (bs, 2H), 1.802 (s, 3H), 1.274-1.291 (d, 3H, 6.8 Hz), 0.84 (m, 1H), 0.554-0.598 (m, 2H), 0.326-0.338 (m, 2H) ppm. [M+H] m/z 463.7.

What is claimed is:

1. A compound of Formula 1: 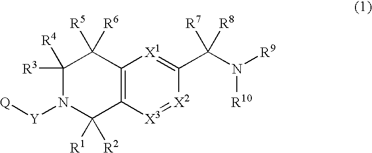
wherein:
each X¹, X², and X³ are each, independently, CR¹¹ or nitrogen;
Y is a direct bond, —C(O)—, —O—, —(CR¹²R¹³)ₘ—, —NR²⁰—, or —S(O)_—;
Q is selected from the group consisting of aryl, heteroaryl, cycloalkyl, heterocycloalkyl or heterocycloalkenyl group; wherein aryl, heteroaryl, cycloalkyl, heterocycloalkyl and heterocycloalkenyl is optionally substituted with one or more substituents selected from the group consisting of halogen, alkyl, heteroalkyl, —CN, —NO₂, —Si(R¹¹)₃, —S(O)ₙR¹¹, —C(O)H, —C(O)R¹⁴, —NR¹¹R¹⁴, C(O)OR¹⁴, NHS(O)ₙCH₃, OR¹¹, SR¹⁴, lower alkoxy, fluoro substituted lower alkoxy, lower alkylthio, fluoro substituted lower alkylthio, and wherein said aryl, heteroaryl, cycloalkyl, and heterocycloalkyl groups are optionally fused with or covalently bound to other aryl, heteroaryl, cycloalkyl, or heterocycloalkyl groups to form a polycyclic ring system having 2 to 4 rings;
R¹, R², R³, R⁴, R⁵, R⁶, R¹², and R¹³ are, independently, selected from the group consisting of hydrogen, halogen, alkyl, heteroalkyl, cycloalkyl, heterocycloalkyl, arylalkyl, heteroarylalkyl, —CN, —OR¹⁵, —NO₂, —NR¹¹R¹⁴, —SiR²⁰₃, —S(O)ₙR¹⁶, —C(O)H, and —C(O)R¹⁷;
R⁷ and R⁸ are each, independently, selected from the group consisting of hydrogen, halogen, alkyl, heteroalkyl, cycloalkyl, heterocycloalkyl, —CN, —OR¹⁵, —NO₂, —SiR²⁰₃, —NR⁹R¹⁰, —S(O)ₙR¹⁶, —C(O)H, and —C(O)R¹⁷;
R⁹ and R¹⁰ are each, independently, selected from the group consisting of hydrogen, alkyl, heteroalkyl, cycloalkyl, heterocycloalkyl, aryl, heteroaryl, arylalkyl, heteroarylalkyl, —S(O)ₙR¹⁶, —C(O)H, —C(O)R¹⁷, and —NR¹⁸R¹⁹;
R¹¹ is hydrogen, halogen, alkylene, alkyl, heteroalkyl, cycloalkyl, heterocycloalkyl, —CN, —OR¹⁵, —NO₂, —SiR²⁰₃, —N(R¹⁴)₂, —S(O)ₙR¹⁶, —C(O)H, and —C(O)R¹⁷, wherein alkylene is optionally substituted with one or more substituents selected from alkyl, cycloalkyl, heteroalkyl, heteroaryl, heterocycloalkyl, —N(R¹⁴)ₙ, —OR¹⁴, or —C(O)OR¹⁴;
R¹⁴ is alkyl, alkoxy, heteroalkyl, cycloalkyl, heterocycloalkyl, aryl, heteroaryl, arylalkyl, heteroarylalkyl, —C(O)H, —C(O)R¹⁷, or —NR¹⁸R¹⁹;
R¹⁵ can be the same or different and is selected from the group consisting of hydrogen, alkyl, heteroalkyl, cycloalkyl, heterocycloalkyl, aryl, heteroaryl, arylalkyl, heteroarylalkyl, and —P(O)₂OR²⁰;
R¹⁶ is alkyl, heteroalkyl, cycloalkyl, heterocycloalkyl, aryl, heteroaryl, arylalkyl, or heteroarylalkyl;
R¹⁷ is —OR¹⁶, —SR¹⁶, —NR¹⁸R¹⁹, alkyl, heteroalkyl, cycloalkyl, heterocycloalkyl, aryl, heteroaryl, arylalkyl, or heteroarylalkyl;
each R¹⁸ and R¹⁹ are, independently, selected from the group consisting of hydrogen, alkyl, heteroalkyl, cycloalkyl, heterocycloalkyl, aryl, heteroaryl, arylalkyl, and heteroarylalkyl;
R²⁰ is hydrogen, alkyl, heteroalkyl, cycloalkyl, heterocycloalkyl, aryl, heteroaryl, arylalkyl, or heteroarylalkyl;
m is 1 or 2; and
each n is, independently 0, 1, or 2;
or optical isomers, pharmaceutically acceptable salts, solvates or N-oxides thereof;
with the proviso that at least one of R⁷ and R⁸ is not both hydrogen when Y is —(CR¹²R¹³)ₘ—.

2. A compound of Formula 1: 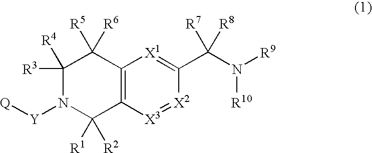
wherein:
each X¹, X², and X³ are each, independently, CR¹¹ or nitrogen;
Y is a direct bond, —C(O)—, —O—, —(CR¹²R¹³)ₘ—, —NR²⁰—, or —S(O)ₙ—;
Q is selected from the group consisting of aryl, heteroaryl, cycloalkyl, heterocycloalkyl or heterocycloalkenyl group; wherein aryl, heteroaryl, cycloalkyl, heterocycloalkyl and heterocycloalkenyl is optionally substituted with one or more substituents selected from the group consisting of halogen, alkyl, heteroalkyl, —CN, —NO₂, —Si(R¹¹)₃, —S(O)ₙR¹¹, —C(O)H, —C(O)R¹⁴, —NR¹¹R¹⁴, C(O)OR¹⁴, NHS(O)ₙCH₃, OR¹¹, SR¹⁴, lower alkoxy, fluoro substituted lower alkoxy, lower alkylthio, fluoro substituted lower alkylthio, and wherein said aryl, heteroaryl, cycloalkyl, and heterocycloalkyl groups are optionally fused with or covalently bound to other aryl, heteroaryl, cycloalkyl, or heterocycloalkyl groups to form a polycyclic ring system having 2 to 4 rings;
R¹, R², R³, R⁴, R⁵, R⁶, R¹², and R¹³ are, independently, selected from the group consisting of hydrogen, halogen, alkyl, heteroalkyl, cycloalkyl, heterocycloalkyl, arylalkyl, heteroarylalkyl, —CN, —OR¹⁵, —NO₂, —NR¹¹R¹⁴, —SiR²⁰₃, —S(O)ₙR¹⁶, —C(O)H, and —C(O)R¹⁷; and wherein geminal R¹, R², R³, R⁴, R⁵, R⁶, R¹², or R¹³ groups can, independently, form carbonyl, thiocarbonyl, spiro-cyclopropyl, substituted imines or oximes with the carbon atom to which they are bound;
R⁷ and R⁸ are each, independently, selected from the group consisting of hydrogen, halogen, alkyl, heteroalkyl, cycloalkyl, heterocycloalkyl, —CN, —OR¹⁵, —NO₂, —SiR²⁰₃, —NR¹¹R¹⁴, —S(O)ₙR¹⁶, —C(O)H, and —C(O)R¹⁷; wherein geminal R⁷ and R⁸ groups can, independently, form spiro-cyclopropyl with the carbon atom to which they are bound; and wherein R⁷ and R⁸ do not collectively form a carbonyl with the carbon atom to which they are bound;
R⁹ and R¹⁰ are each, independently, selected from the group consisting of hydrogen, alkyl, heteroalkyl, cycloalkyl, heterocycloalkyl, aryl, heteroaryl, arylalkyl, heteroarylalkyl, —S(O)ₙR¹⁶, —C(O)H, —C(O)R¹⁷, and —NR¹⁸R¹⁹; wherein R⁹ and R¹⁰ groups can form heteroaryl groups with the nitrogen atom to which they are bound; wherein R⁹ and R¹⁰ groups can form heterocycloalkyl groups with the nitrogen atom to which they are bound when R⁷ and R⁸ are not both hydrogen; wherein R⁹ and R¹⁰ groups can, independently, form heterocycloalkyl groups with the nitrogen atom to which they are bound when at least one of X¹, X², or X³ is nitrogen; wherein R⁹ is not —CH(alkyl)C(O)NH₂ when R¹⁰ is —S(O)ₙR¹⁶ and n is 2 and R¹⁶ is a substituted or unsubstituted phenyl or thiophene group; and wherein R⁹ is not methyl when R¹⁰ is pyrrolidin-3-yl;
R¹¹ is hydrogen, halogen, alkylene, alkyl, heteroalkyl, cycloalkyl, heterocycloalkyl, —CN, —OR¹⁵, —NO₂, —SiR²⁰₃, —N(R¹⁴)ₙ, —S(O)ₙR¹⁶, —C(O)H, or —C(O)R¹⁷, wherein alkylene is optionally substituted with one or more substituents selected from the group consisting of alkyl, cycloalkyl, heteroalkyl, heteroaryl, heterocycloalkyl, —N(R¹⁴)ₙ, —OR¹⁴, and —C(O)OR¹⁴;
R¹⁴ is alkyl, alkoxy, heteroalkyl, cycloalkyl, heterocycloalkyl, aryl, heteroaryl, arylalkyl, heteroarylalkyl, —C(O)H, —C(O)R¹⁷, or —NR¹⁸R¹⁹;
R¹⁵ can be the same or different and is hydrogen, alkyl, heteroalkyl, cycloalkyl, heterocycloalkyl, aryl, heteroaryl, arylalkyl, heteroarylalkyl, or —P(O)₂OR²⁰;
R¹⁶ is alkyl, heteroalkyl, cycloalkyl, heterocycloalkyl, aryl, heteroaryl, arylalkyl, or heteroarylalkyl;
R¹⁷ is —OR¹⁶, —SR¹⁶, —NR¹⁸R¹⁹, alkyl, heteroalkyl, cycloalkyl, heterocycloalkyl, aryl, heteroaryl, arylalkyl, or heteroarylalkyl;
each R¹⁸ and R¹⁹ is, independently, selected from the group consisting of hydrogen, alkyl, heteroalkyl, cycloalkyl, heterocycloalkyl, aryl, heteroaryl, arylalkyl, and heteroarylalkyl; and wherein geminal R¹⁸ and R¹⁹ groups can, independently, form heteroaryl or heterocycloalkyl groups with the nitrogen atom to which they are bound;
R²⁰ is hydrogen, alkyl, heteroalkyl, cycloalkyl, heterocycloalkyl, aryl, heteroaryl, arylalkyl, or heteroarylalkyl;
m is 1 or 2; and
each n is, independently 0, 1, or 2;
or optical isomers, pharmaceutically acceptable salts, solvates or N-oxides thereof;
with the proviso that at least one of R⁷ and R⁸ is not both hydrogen when Y is —(CR¹²R¹³)ₘ—.

3. The compound of claim 1, wherein Q is an aryl group, and said aryl is optionally substituted with one or more substituents selected from the group consisting of halogen, alkyl, heteroalkyl, —CN, —NO₂, —Si(R¹¹)₃, —S(O)ₙR¹¹, —C(O)H, —C(O)R¹⁴, —NR¹¹R¹⁴, C(O)OR¹⁴, NHS(O)ₙCH₃, OR¹¹, SR¹⁴, lower alkoxy, fluoro substituted lower alkoxy, lower alkylthio, and fluoro substituted lower alkylthio.

4. The compound of claim 3, wherein Q is selected from the group consisting of: 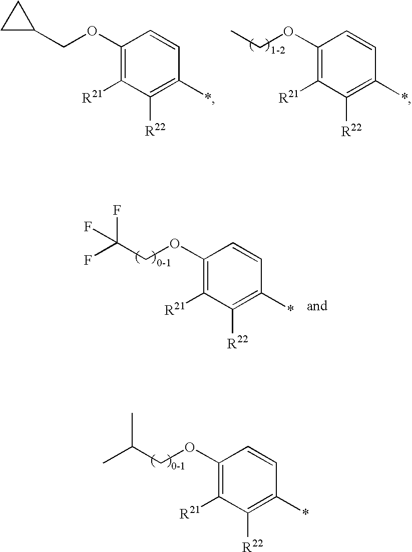
wherein, R²¹, R²² are independently selected from the group consisting of alkyl, —NO₂, fluoro substituted lower alkoxy and halogen.

5. The compound of claim 1, wherein Q is a heteroaryl group, said heteroaryl is optionally substituted with one or more substituents selected from the group consisting of halogen, alkyl, heteroalkyl, —CN, —NO₂, —Si(R¹¹)₃, —S(O)ₙR¹¹, —C(O)H, —C(O)R¹⁴, —NR¹¹R¹⁴, C(O)OR¹⁴, NHS(O)ₙCH₃, OR¹¹, SR¹⁴, lower alkoxy, fluoro substituted lower alkoxy, lower alkylthio, fluoro substituted lower alkylthio.

6. The compound of claim 1, wherein Q is a cycloalkyl group, and said cycloalkyl is optionally substituted with one or more substituents selected from the group consisting of halogen, alkyl, heteroalkyl, —CN, —NO₂, —Si(R¹¹)₃, —S(O)ₙR¹¹, —C(O)H, —C(O)R¹⁴, —NR¹¹R¹⁴, C(O)OR¹⁴, NHS(O)ₙCH₃, OR¹¹, SR¹⁴, lower alkoxy, fluoro substituted lower alkoxy, lower alkylthio, fluoro substituted lower alkylthio.

7. The compound of claim 1, wherein Q is a heterocycloalkyl group, wherein said heterocycloalkyl is optionally substituted with one or more substituents selected from the group consisting of halogen, alkyl, heteroalkyl, —CN, —NO₂, —Si(R¹¹)₃, —S(O)ₙR¹¹, —C(O)H, —C(O)R¹⁴, —NR¹¹R¹⁴, C(O)OR¹⁴, NHS(O)ₙCH₃, OR¹¹, SR¹⁴, lower alkoxy, fluoro substituted lower alkoxy, lower alkylthio, fluoro substituted lower alkylthio.

8. The compound of claim 1, wherein X¹, X², and X³ are CR¹¹.

9. The compound of claim 1, wherein when Y is —C(O)— or —CH₂C(O)—, Q is not a substituted or unsubstituted pyrrolidin-1-yl, piperidin-1-yl, azepan-1-yl, azocan-1-yl, piperazin-1-yl, 1,4-diazepan-1-yl, or 1,4-diazocan-1-yl.

10. The compound of claim 1, wherein Y is —C(O)— or —CH₂—.

11. The compound of claim 1, wherein Y is —CH₂—, Q is an aryl group, wherein said aryl is optionally substituted with one or more substituents selected from the group consisting of halogen, alkyl, heteroalkyl, —CN, —NO₂, —Si(R¹¹)₃, —S(O)ₙR¹¹, —C(O)H, —C(O)R¹⁴, —NR¹¹R¹⁴, C(O)OR¹⁴, NHS(O)ₙCH₃, OR¹¹, SR¹⁴, lower alkoxy, fluoro substituted lower alkoxy, lower alkylthio, and fluoro substituted lower alkylthio.

12. The compound of claim 1, wherein Y is —CH₂—, and Q is selected from the group consisting of aryl, heteroaryl, cycloalkyl, heterocycloalkyl and heterocycloalkenyl group, wherein aryl, heteroaryl, cycloalkyl, heterocycloalkyl, and heterocycloalkenyl is not unsubstituted.

13. The compound of claim 1, wherein when m is 2, then R⁷ and R⁸ are not both hydrogen.

14. The compound of claim 1, wherein geminal R¹, R², R³, R⁴, R⁵, R⁶, R¹², or R¹³ groups, independently, form carbonyl, thiocarbonyl, spiro-cyclopropyl, substituted imines (imines in the form of —C(═NR¹⁴)—), or oximes (oximes in the form of —C(═N—OR²⁰)—) with the carbon atom to which they are bound.

15. The compound of claim 1, wherein geminal R⁷ and R⁸ groups, independently, form spiro-cyclopropyl with the carbon atom to which they are bound.

16. The compound of claim 1, wherein R⁷ and R⁸ do not collectively form a carbonyl with the carbon atom to which they are bound.

17. The compound of claim 1, wherein R⁹ and R¹⁰ groups form heteroaryl groups with the nitrogen atom to which they are bound.

18. The compound of claim 1, wherein R⁹ and R¹⁰ groups form heterocycloalkyl groups with the nitrogen atom to which they are bound when R⁷ and R⁸ are not both hydrogen.

19. The compound of claim 1, wherein R⁹ and R¹⁰ groups, independently, form heterocycloalkyl groups with the nitrogen atom to which they are bound when at least one of X¹, X², or X³ is nitrogen.

20. The compound of claim 1, wherein R⁹ is not —CH(alkyl)C(O)NH₂ when R¹⁰ is —S(O)ₙR¹⁶, n is 2, and R¹⁶ is a substituted or unsubstituted phenyl or thiophene group.

21. The compound of claim 1, wherein R⁹ is not methyl when R¹⁰ is pyrrolidin-3-yl.

22. The compound of claim 1, wherein geminal R¹⁸ and R¹⁹ groups, independently, form heteroaryl or heterocycloalkyl groups with the nitrogen atom to which they are bound.

23. The compound of claim 1, wherein the compound is selected from the group consisting of:
N-{1-[2-(4-Cyclopropylmethoxybenzoyl)-1,2,3,4-tetrahydroisoquinolin-6-yl]ethyl}acetamide,
N-{1-[2-(4-Cyclopropylmethoxybenzyl)-1,2,3,4-tetrahydroisoquinolin-6-yl]ethyl}acetamide,
N-{1-[2-(4-Cyclopropylmethoxybenzyl)-1,2,3,4-tetrahydroisoquinolin-6-yl]ethyl}propionamide,
{1-[2-(4-Cyclopropylmethoxybenzyl)-1,2,3,4-tetrahydroisoquinolin-6-yl]ethyl}carbamic acid methyl ester,
{1-[2-(4-Cyclopropylmethoxybenzyl)-1,2,3,4-tetrahydroisoquinolin-6-yl]ethyl}carbamic acid ethyl ester,
N-{1-[2-(3-Chloro-4-propoxybenzyl)-1,2,3,4-tetrahydroisoquinolin-6-yl]ethyl}acetamide,
N-{1-[2-(3-Chloro-4-isobutoxybenzyl)-1,2,3,4-tetrahydroisoquinolin-6-yl]ethyl}acetamide,
N-{1-[2-(3-Chloro-4-isobutoxybenzyl)-1,2,3,4-tetrahydroisoquinolin-6-yl]ethyl}acetamide,
N-{1-[2-(3-Chloro-4-cyclopropylmethoxybenzyl)-1,2,3,4-tetrahydroisoquinolin-6-yl]ethyl}acetamide,
N-{1-[2-(3-Chloro-4-ethoxybenzyl)-1,2,3,4-tetrahydroisoquinolin-6-yl]ethyl}acetamide,
N-{1-[2-(3-Bromo-4-isopropoxybenzyl)-1,2,3,4-tetrahydroisoquinolin-6-yl]ethyl}acetamide,
N-{1-[2-(3-Bromo-4-propoxybenzyl)-1,2,3,4-tetrahydroisoquinolin-6-yl]ethyl}acetamide,
N-{1-[2-(3-Bromo-4-ethoxybenzyl)-1,2,3,4-tetrahydroisoquinolin-6-yl]ethyl}acetamide,
N-{1-[2-(3-Bromo-4-cyclopropylmethoxybenzyl)-1,2,3,4-tetrahydroisoquinolin-6-yl]ethyl}acetamide,
N-{1-[2-(3-Bromo-4-isobutoxybenzyl)-1,2,3,4-tetrahydroisoquinolin-6-yl]ethyl}acetamide,
N-{1-[2-(4-Isopropoxy-2-methylbenzyl)-1,2,3,4-tetrahydroisoquinolin-6-yl]ethyl}acetamide,
N-{1-[2-(4-Ethoxy-2-methylbenzyl)-1,2,3,4-tetrahydroisoquinolin-6-yl]ethyl}acetamide,
N-{1-[2-(2-Methyl-4-propoxybenzyl)-1,2,3,4-tetrahydroisoquinolin-6-yl]ethyl}acetamide,
4-[2-(4-Cyclopropylmethoxy-2-methylbenzyl)-1,2,3,4-tetrahydroisoquinolin-6-yl]-pentan-2-one,
N-{1-[2-(4-isopropoxy-2-methylbenzyl)-1,2,3,4-tetrahydroisoquinolin-6-yl]ethyl}acetamide,
N-{1-[2-(2-Bromo-4-isopropoxybenzyl)-1,2,3,4-tetrahydroisoquinolin-6-yl]ethyl}acetamide,
N-{1-[2-(2-Chloro-4-isopropoxybenzyl)-1,2,3,4-tetrahydroisoquinolin-6-yl]ethyl}acetamide,
N-{1-[2-(4-Cyclopropylmethoxybenzyl)-1-oxo-1,2,3,4-tetrahydroisoquinolin-6-yl]ethyl}acetamide,
N-(1-{2-[2-Chloro-4-(2,2,2-trifluoro-ethoxy)-benzyl]-1,2,3,4-tetrahydro-isoquinolin-6-yl}-ethyl)-acetamide,
N-(1-{2-[2-Nitro-4-(2,2,2-trifluoro-ethoxy)-benzyl]-1,2,3,4-tetrahydro-isoquinolin-6-yl}-ethyl)-acetamide,
N-{1-[2-(4-Ethoxy-2-nitro-benzyl)-1,2,3,4-tetrahydro-isoquinolin-6-yl]-ethyl}-acetamide, and
N-{1-[2-(4-cyclopropylmethoxy-3-trifluoromethoxybenzyl)-1,2,3,4-tetrahydroisoquinolin-6-yl]ethyl}acetamide,
or optical isomers, pharmaceutically acceptable salts, solvates or N-oxides thereof.

24. The compound of claim 1, wherein Y is —O—, —NR²⁰—, or —S(O)ₙ—.

25. The compound of claim 1, wherein when m is 2, then (CR¹²R¹³)ₘ, is optionally a 3-, 4-, or 5-membered carbocycle selected from the group consisting of:

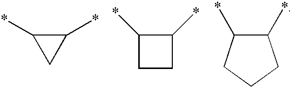

26. The compound of claim 26, wherein said carbocycle is optionally substituted by one or more groups that are, independently, selected from the group consisting of alkyl, halogen, —CN, —OR¹⁵, —SiR²⁰₃, and —NO₂.

27. The compound of claim 1, wherein the compound is an acetyl-CoA carboxylase (ACC) inhibitor.

28. A pharmaceutical composition comprising the compound of claim 1 and a pharmaceutically acceptable carrier.

29. A method for preventing or treating a condition that responds to an acetyl-CoA carboxylase inhibitor, comprising administering to a patient in need thereof an effective amount of a composition according to claim 28.

30. The method of claim 29, wherein the condition is selected from type 2 diabetes, obesity, diabesity, atherosclerosis, and cardiovascular diseases.
